SEQUENCE LISTING (1 sequence)

SEQ ID NO: 1 - protein, 6 aa, Artificial Sequence
  Synthetic
GXGRGX

(19) United States
(12) Patent Application Publication
(10) Pub. No.: US 2017/0199175 A1
(43) Pub. Date: Jul. 13, 2017

(54) DEVICE AND METHOD FOR ASSESSING CELL CONTRACTION

(71) Applicants: The Governing Council of the University of Toronto, Toronto (CA); École Polytechnique Fédérale de Lausanne, Lausanne (CH)

(72) Inventors: Boris HINZ, Toronto (CA); Pierre-Jean WIPFF, Lausanne (CH)

(73) Assignees: The Governing Council of the University of Toronto, Toronto, ON (CA); École Polytechnique Fédérale de Lausanne, Lausanne (CH)

(21) Appl. No.: 15/313,984

(22) PCT Filed: May 26, 2015
(86) PCT No.: PCT/CA2015/000335

§ 371 Date: Nov. 25, 2016

Related U.S. Application Data
(60) Provisional application No. 62/002,975, filed on May 26, 2014.

(51) Int. Cl. G01N 33/50; A61K 35/33; C12M 1/00; G01N 33/483; C12M 1/32

(52) U.S. Cl. CPC G01N 33/5026; G01N 33/4833; C12M 23/12; C12M 23/22; A61K 35/33; G01N 2500/10

(57) ABSTRACT

A method for assessing cell contraction includes adhering contractile cells to an oxidized and cellular adhesion activated surface of a biocompatible silicone elastomer film. The biocompatible silicone elastomer film allows the cells to contract and wrinkles when the cells contract. Wrinkles in the biocompatible silicone elastomer film formed by contraction of the contractile cells are analyzed to assess cell contraction.

### CROSS REFERENCES TO RELATED APPLICATIONS

This application claims priority from U.S. provisional patent application No. 62/002,975 filed on May 26, 2014, which is incorporated herein by reference in its entirety.

### FIELD

The disclosure relates to devices and methods for assessing cell contraction. In particular, the disclosure relates to devices incorporating wrinkling films, and methods for using wrinkling films to assess cell contraction.

### BACKGROUND

U.S. Patent Application Publication No. 2009/0186411 (Hoffmann et al.) purports to disclose a cell culture apparatus for cells, which includes a surface composed of an unstructured elastomer. The cells are cultured under close to natural conditions in relation to their environmental elasticity. A method for producing an apparatus according to the invention is purportedly disclosed, as is a cell culture method using such an apparatus.

PCT Patent Application Publication No. WO/2009/032164 (Tschumperlin et al.) purports to disclose a multi-well plate that can be loaded with a range of compliant substrates. Commercially available assays can be used to test cellular responses across a plate with shear modulus from 50 to 51200 Pascals. Cells can be grown in the plates, and can be manipulated and analyzed. Hydrogels can be attached to the bottom of a well. The plates can support the attachment and growth of different cell types and can be compatible with standard 96-well and 384-well plate assays. The mechanical properties of the hydrogels can be reproducible and stable to increase the shelf life of the substrate. The hydrogel can be compatible with growth of a variety of cell types, various attachment ligands such as collagen I, collagen IV, fibronectin, vitronectin, laminin, or RGD peptides and can be coupled to the gel surface.

PCT Patent Application Publication No. WO 2013/074972 (Butler et al.) purports to disclose a platform for biological assays that includes a base substrate providing structural support to the platform, and at least one surface of the base substrate coated with position markers. A first deformable layer is positioned on top of the base substrate, and a second deformable layer is positioned on top of the first deformable layer. The second deformable layer is embedded with deformation markers.

### SUMMARY

The following summary is intended to introduce the reader to various aspects of the disclosure, but not to define any invention.

According to one aspect, a method for assessing cell contraction comprises a) adhering contractile cells to an oxidized and cellular adhesion activated surface of a biocompatible silicone elastomer film. The biocompatible silicone elastomer film allows the cells to contract and wrinkles when the cells contract. The method further comprises b) analyzing the wrinkles in the biocompatible silicone elastomer film formed by contraction of the contractile cells.

The contractile cells may include at least one of fibroblasts, myofibroblasts, epithelial cells, endothelial cells, cardiomyocytes, skeletal muscle cells, smooth muscle cells, mesenchymal stem cells, induced pluripotent stem cells, embryonic stem cells, inflammatory cells, cancer cells, immortalized lineage cells, hepatic stellate cells, pericytes, chondrocytes, chondroblasts, osteoblasts, osteoclasts, astrocytes, myoepithelial cells, glial cells, and neuronal cells. In one specific example, the contractile cells may be cardiomyocytes. In another specific example, the contractile cells may be fibroblasts.

The cells may be in the form of a tissue. The tissue may be at least one of fibrotic tissue, scar tissue, heart muscle tissue, skeletal muscle tissue, smooth muscle tissue, arterial tissue, venous tissue, connective tissue, nervous tissue, liver tissue, kidney tissue, lung tissue, gastrointestinal tissue, cancer tissue, bone marrow tissue, blood tissue, cartilage tissue, bone tissue, gingiva tissue, skin tissue, tendon tissue, fascia tissue, glandular tissue, embryonic tissue, and reproductive tissue.

The biocompatible silicone elastomer film may be fully polymerized.

The biocompatible silicone elastomer film may comprise a polydimethylsiloxane.

Step b) may comprise obtaining an image of the biocompatible silicone elastomer film, determining a proportion of the image that contains the wrinkles, and comparing the proportion to a control. Step b) may be performed via live imaging. Step b) may be performed in real time.

Prior to step a), the method may further comprise oxidizing the surface of a raw biocompatible silicone elastomer film, and activating the oxidized surface for cellular adhesion, to yield the oxidized and cellular adhesion activated surface. Oxidizing the surface may comprise plasma oxidation of the surface. Alternatively, oxidizing the surface may comprise treating the surface with hydrogen peroxide and sulfuric acid.

Activating the oxidized surface for cellular adhesion may comprise silanizing the oxidized surface, and treating the oxidized surface with an extracellular matrix (ECM) protein.

The method may further comprise fluorescently labeling the ECM protein.

Silanizing the oxidized surface may comprise treating the oxidized surface with 3-aminopropyltriethoxysilane (APTES). In some examples, silanizing the oxidized surface may further comprise treating the surface with at least one of paraformaldehyde and 1-ethyl-3-(3-dimethylaminopropyl) carbodiimide (EDAC) subsequent to treatment with APTES. In some examples, Silanizing the oxidized surface may further comprise treating the surface with reactive fluorochromes subsequent to treatment with APTES. In some specific examples, the reactive moiety of the fluorochrome may comprise isotyocyanate (ITC) and the fluorochrome may comprise rhodamine (Rh). In some specific examples, the fluorochromes may comprise Alexa®-based dyes.

The ECM protein may include at least one of gelatin, collagen, fibronectin, vitronectin, pronectin, DOPA, N-acetyl glucosamine, laminin, bovine serum albumin (BSA), RGD peptides and derivatives, and combinations thereof. In some specific examples, the ECM protein may be gelatin. In some specific examples, the ECM protein may be collagen. In some specific examples, the ECM protein may be fibronectin.

Prior to step b), the method may further comprise treating the cells to induce contraction or inhibit contraction.

Prior to step b), the method may further comprise treating the cells with a test compound. Step b) may comprise assessing the effect of the test compound on cell contraction. The method may further comprise identifying the test compound as an inducer of contraction or an inhibitor of contraction. If the test compound is identified as an inducer of contraction, the method may comprise selecting the test compound as a candidate treatment for at least one of chronic wound healing, low vascular tone, arrhythmia, and muscular dystrophy. If the test compound is identified as an inhibitor of contraction, the method may comprise selecting the test compound as a candidate treatment for at least one of fibrocontractive disease, and cancer. If the test compound is identified as an inhibitor of contraction, the method may also comprise selecting the test compound as a candidate smooth and skeletal muscle relaxant.

The method may further comprise selecting highly contractile cells of the contractile cells based on the analysis of step b), for purposes of autologous cell selection for cell therapies. The highly contractile cells may be transplanted into a patient for cell therapy.

The film may comprise a fluorescent dye, and step b) may comprise imaging the wrinkles with fluorescence microscopy.

The contractile cells may be cardiomyocytes, and step b) may comprise quantifying a percentage of the contractile cells that are beating, and/or determining a beating rate of the cells. Step b) may also comprise determining a contractile force of the cells.

The method may further comprise blending a cell adhesive peptide coupled to a bioactive fluorinated surface modifier (BFSM) into the biocompatible silicone elastomer film.

The method may further comprise embedding a position marker in the biocompatible silicone elastomer film.

The biocompatible silicone elastomer film may have a modulus of elasticity of between 0.5 kPa and 25 kPa. In one example, the biocompatible silicone elastomer film may have a modulus of elasticity of about 5 kPa. In another example, the biocompatible silicone elastomer film may have a modulus of elasticity of between about 1.5 and 3.0 kPa.

The film may have a film thickness of less than 200 microns, more specifically between 20 microns and 40 microns, and more specifically of approximately 30 microns.

According to another aspect, a device for assessing cell contraction comprises a plate comprising at least one well. Each well has a well sidewall and a planar well bottom. Each well bottom comprises a coating of a biocompatible silicone elastomer film having an oxidized and cellular adhesion activated surface.

The film may have a film thickness of less than 200 microns, more specifically between 20 microns and 40 microns, and more specifically of approximately 30 microns.

The plate may comprise an upper plate comprising at least one bottomless well. Each bottomless well may define at least one well sidewall. A base plate may be formed separately from the upper plate and secured to the upper plate. The base plate may comprise a planar face coated with the biocompatible silicone elastomer film to form the well bottom of each well.

The base plate may have a thickness of between 100 microns and 200 microns, more specifically of about 150 microns.

The base plate may be transparent, for example may be fabricated from glass or plastic.

The upper plate may be fabricated from polystyrene.

The plate may comprise a plurality of wells. For example, the plate may comprise 96 wells. Alternatively, the plate may comprise 384 wells.

The biocompatible silicone elastomer film may comprise a polydimethylsiloxane.

The oxidized and cellular adhesion activated surface may comprise an extracellular matrix (ECM) protein and the ECM protein may be at least one of gelatin, collagen, fibronectin, vitronectin, pronectin, DOPA, N-acetyl glucosamine, BSA, laminin, RGD peptides and derivatives, and combinations thereof. The ECM protein may be fluorescently labeled. In some examples, the ECM protein may be gelatin. In some examples, the ECM protein may be collagen.

The biocompatible silicone elastomer film may comprise a fluorescent dye.

The device may further comprise at least one position marker in the biocompatible silicone elastomer film.

The biocompatible silicone elastomer film may have a modulus of elasticity of between 0.5 kPa and 25 kPa. In one example, the biocompatible silicone elastomer film may have a modulus of elasticity of about 5 kPa. In another example, the biocompatible silicone elastomer film may have a modulus of elasticity of between about 1.5 and 3.0 kPa.

The biocompatible silicone elastomer film may be transparent.

According to another aspect, a method for assessing cell contraction using the above device comprises a) adhering contractile cells to at least one of the oxidized and cellular adhesion activated surfaces. The biocompatible silicone elastomer film allows the cells to contract and wrinkles when the cells contract. The method further comprises b) analyzing wrinkles in the biocompatible silicone elastomer film formed by contraction of the contractile cells.

According to another aspect, a method for fabricating a cell contraction assessment device comprises a) coating a planar face of a base plate with a raw biocompatible silicone elastomer film; b) oxidizing a surface of the raw biocompatible silicone elastomer film, and activating a surface of the oxidized biocompatible silicone elastomer film for cellular adhesion, to yield a biocompatible silicone elastomer film having an oxidized and cellular adhesion activated surface; and c) securing the base plate to an upper plate comprising at least one bottomless well. The at least one bottomless well and base plate together form at least one well. Each well has a well sidewall formed by one of the bottomless wells of the upper plate, and a well bottom formed by the base plate and the biocompatible silicone elastomer film.

Step a) may comprise coating the planar face of the base plate with the raw biocompatible silicone elastomer film to yield a film thickness of less than 200 microns, more specifically between 20 microns and 40 microns, more specifically approximately 30 microns.

Step b) may comprise plasma oxidation of the surface of the raw biocompatible silicone elastomer film. Alternatively, step b) may comprise treating the surface of the raw biocompatible silicone elastomer film with hydrogen peroxide and sulfuric acid.

Step b) may comprise silanizing the surface of the oxidized biocompatible silicone elastomer film; and treating the surface of the oxidized biocompatible silicone elastomer film with an extracellular matrix (ECM) protein.

The ECM protein may include at least one of gelatin, collagen, fibronectin, vitronectin, pronectin, DOPA, N-acetyl glucosamine, BSA, laminin, RGD peptides and derivatives, and combinations thereof. In some specific examples, the ECM protein may be collagen. In some specific examples, the ECM protein may be gelatin. The method may further comprise fluorescently labeling the ECM protein.

Silanizing the surface of the oxidized biocompatible silicone elastomer film may comprise treating the oxidized biocompatible silicone elastomer film with 3-aminopropyltriethoxysilane (APTES). Silanizing the surface of the oxidized biocompatible silicone elastomer film may further comprise treating the oxidized surface of the raw biocompatible silicone elastomer film with at least one of paraformaldehyde and 1-ethyl-3-(3-dimethylaminopropyl) carbodiimide (EDAC) subsequent to treatment with APTES.

Step b) may comprise treating the oxidized surface with reactive fluorochromes subsequent to treatment with APTES. In some specific examples, the reactive moiety of the fluorochrome may comprise isotyocyanate (ITC) and the fluorochrome may comprise rhodamine (Rh). In some specific examples, the fluorochromes may comprise Alexa®-based dyes.

Step b) may comprise blending a cell adhesive peptide coupled to a bioactive fluorinated surface modifier (BFSM) into the raw biocompatible silicone elastomer film.

The raw biocompatible silicone elastomer film may comprise a polydimethylsiloxane.

The base plate may have a thickness of between 100 microns and 200 microns, more specifically about 150 microns.

The base plate may be transparent. The base plate may be fabricated from glass or plastic.

The upper plate may be fabricated from polystyrene.

The upper plate may comprise a plurality of wells. The upper plate may comprise 96 bottomless wells. The upper plate may comprise 384 bottomless wells.

Step c) may comprise clamping the upper plate to the base plate.

Step a) may comprise spin casting the raw biocompatible silicone elastomer film onto the planar face.

The method may further comprise embedding a position marker in the raw biocompatible silicone elastomer film.

The method may further comprise incorporating a fluorescent dye into the raw biocompatible silicone elastomer film.

The biocompatible silicone elastomer film may have a modulus of elasticity of between 0.5 kPa and 25 kPa. In one example, the biocompatible silicone elastomer film may have a modulus of elasticity of about 5 kPa. In another example, the biocompatible silicone elastomer film may have a modulus of elasticity of between about 1.5 kPa and about 3.0 kPa.

The biocompatible silicone elastomer film may be transparent.

### BRIEF DESCRIPTION OF THE DRAWINGS

The drawings included herewith are for illustrating various examples of articles, methods, and apparatuses of the present specification and are not intended to limit the scope of what is taught in any way. In the drawings:

FIG. 1 shows fibroblast generated wrinkles in a biocompatible silicone elastomer film having an oxidized and cellular adhesion activated surface, as captured by atomic force microscopy (AFM) imaging mode (left, middle), and conventional white light phase contrast microscopy at low magnification (10×) (right), where wrinkles are visible as white lines that are clearly distinct from the cell background;

FIG. 2 is a perspective view of an example device for measuring cell contraction;

FIG. 3 is an exploded perspective view of the device of FIG. 2;

FIG. 4 is a cross section taken along line 4-4 in FIG. 2;

FIG. 5 shows images of rat lung fibroblasts that were grown for 1 day on biocompatible silicone elastomer films (PDMS) that were subject to different surface treatments to improve wrinkle morphology and cell adhesion. Contractile primary rat lung myofibroblasts grown for 1 d generated surface wrinkles that differed morphologically in phase contrast images, depending on whether the surface was treated with collagen alone (protein only), oxidized via plasma oxidation and activated for cellular adhesion with collagen (plasma+protein), oxidized with sulfuric acid and activated for adhesion with APTES, paraformaldehyde, and collagen (H₂SO₄+APTES+protein), or oxidized via plasma oxidation and activated for cellular adhesion with APTES, paraformaldehyde, and collagen (plasma+APTES+protein).

FIG. 6 relates to biocompatible silicone elastomer films that were produced with a polydimethylsiloxane (PDMS) curing agent-to-base ratio of 1:100 and used as culture substrates after oxidation via plasma oxidation and activation for cellular adhesion with APTES, paraformaldehyde, and collagen. Substrates were completely elastic and thus allowed detection of (a-b) increased cell contraction in response to lysophosphatic acid (LPA) by de novo appearance of wrinkles (arrowheads) and (c) loss of cell contraction after cytochalasin D treatment by reduced wrinkle size and number. (d) Corresponding kymograph analysis along the indicated line facilitated analysis of wrinkle development over time (t) of treatment with 10 μM LPA (L, added at t=0), 1 μM (C₁) and 10 μM (C₂) Cytochalasin D and after washing (W). Changes of conditions are indicated by white lines left of the characters. Converging dotted lines highlight approaching wrinkles, i.e. cell contraction whereas diverging wrinkles indicate cell relaxation.

FIG. 7 relates to elastic properties of biocompatible silicone elastomer films. (a) The Young's modulus of PDMS samples produced with curing agent-to-base ratios from 1:110 to 1:40 was calculated from the dynamic shear modulus determined with a rheometer. Mean values ±SD were obtained from three independent samples per condition, each tested 5-times. (b) To evaluate the influence of different surface treatment, PDMS surface stiffness was probed on the cell level using AFM (atomic force microscopy) with spherical-tipped cantilevers. Substrate topography (c) and force indentation profile (d) produced with AFM on a 100×100 μm area is compared between 1:70 and 1:110 substrates surfaces. (c) The elastic modulus was fitted with a conventional Hertz sphere model from force-indentation curves; ten regions were probed per sample performed in triplicates and expressed as mean values ±SD. (d) The minimum force required to wrinkle chemically activated PDMS was determined using microneedles and is displayed as a function of the substrate's Young's modulus. Y-axis error bars indicate SD of mean of measurements, x-axis bars consider 5% error to due to pipetting uncertainties of the viscous polymer. In FIG. 7, ‘collagen coating’ refers to treatment with collagen alone, ‘acid functionalization’ refers to oxidation with sulfuric acid, and ‘plasma activation’ refers to plasma oxidation.

FIG. 8 shows a comparison of the contractile activity of different cell types from their capacity to wrinkle silicone substrates with increasing stiffness. Wrinkling biocompatible silicone elastomer films having a surface that was oxidized with sulfuric acid and activated for adhesion with APTES, paraformaldehyde, and collagen type I were produced. Phase contrast pictures were taken of (a) rat smooth muscle cells (b), lung myofibroblast, (c) and subcutaneous fibroblasts after 1 d culture. (d) The percentage of wrinkling cells was manually determined from 10 image fields per substrate, performed in triplicates and is displayed ±SD as a function of substrate compliance. Note the decrease of wrinkling cells on stiffer substrates, which was more pronounced in low contractile cell types.

FIG. 9 shows that wrinkles were preserved after chemical fixation and immunostaining. (a) Wrinkling biocompatible silicone elastomer films having a surface that was oxidized with sulfuric acid and activated for cellular adhesion with APTES, paraformaldehyde, and collagen type I were produced and used as culture substrate for contractile lung fibroblasts. (b) After 1 d culture, cells were fixed with paraformaldehyde (PFA) (arrow) for 10 min during phase contrast live videomicroscopy. (c) Kymograph analysis along the indicated line demonstrates that only few wrinkles (b, arrowheads) disappear and that cells only slightly relax during fixation. (d) The same cell was then immunostained for α-SMA and F-actin, allowing colocalization of proteins with wrinkles. (e) In 5-10% of all fixed cells, the elastic tension stored in the film wrinkle leads to breakage (arrowheads) of stress fibers in close vicinity to the wrinkle (f).

FIG. 10 shows that the elasticity of thick wrinkling substrates induces phenotypic changes in long-term culture. Wrinkling silicone substrates with chemically cross-linked collagen type I (i.e. treated with plasma, APTES and paraformaldehyde) were produced with a Young's modulus of 3 kPa, 9 kPa, 16 kPa, and 47 kPa and a layer thickness of 200 μm to be used as culture substrate for contractile lung myofibroblasts. Cells were fixed and immunostained for α-smooth muscle actin (α-SMA) and F-actin (phalloidin) either after (a) 1 d or (b) 7 d culture. Immunofluorescence images were overlaid with phase contrast wrinkle images. (c) Western blotting (d) and quantification of Western blotting shows that 7 d culture affected expression of the contractile cell marker α-SMA whereas substrates have no influence on α-SMA expression after 1 d. (e) Reduction of α-SMA expression correlates with reduced cell contraction over time. Scale Bar: 50 μm.

FIG. 11 shows that wrinkle observation was possible with live videomicroscopy using green fluorescence protein (GFP) transfected fibroblasts. Images show fibroblasts grown on wrinkling substrates having an oxidized and cellular adhesion activated surface (plasma, APTES, paraformaldehyde, gelatin) (a) Rat embryonic fibroblasts were transfected with a GFP fusion protein of the cell-matrix adhesion protein β3 integrin and the fluorescence signal was overlaid with the phase contrast wrinkling image before (left) and after treatment of the cells with a relaxing drug. (b) Human cardiac fibroblasts were co-transfected with GFP and shRNA. Non-targeting shRNA (left) has no effect on wrinkling whereas shRNA targeting the focal adhesion protein kindlin-2 (right) leads to cell relaxation and loss of wrinkles.

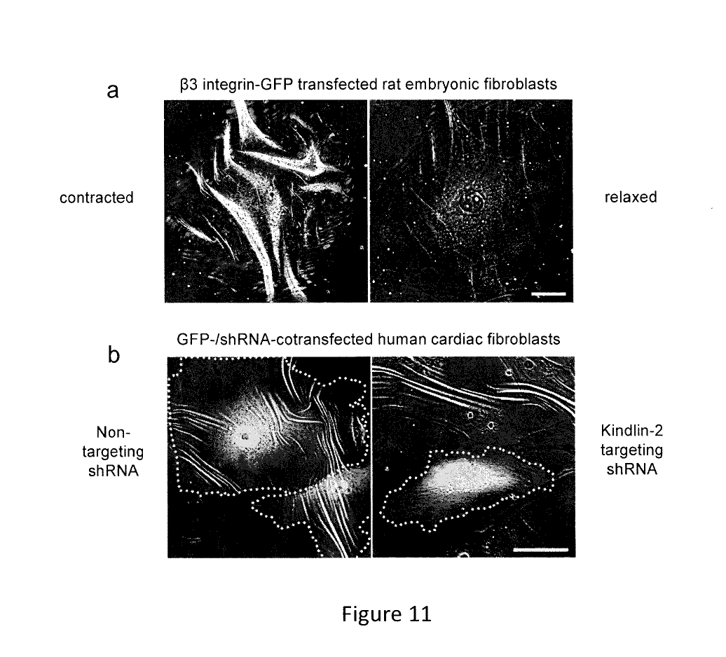

FIG. 12 is a photograph of a device for assessing cell contraction.

FIG. 13 shows that by adjusting the rotation speed of spin-casting of biocompatible silicone elastomer onto support glass coverslips, the thickness of the biocompatible silicone elastomer film was reduced from 200 μm to 30 μm. Thickness measurements performed at the edges and in the center of the coverslips demonstrated even thickness across the whole surface. Thinner substrates have improved optical quality shown by growing fibroblasts on substrates having an oxidized and cellular adhesion activated surface (plasma, APTES, paraformaldehyde, gelatin).

FIG. 14 shows fibroblasts grown on wrinkling substrates having an oxidized and cellular adhesion activated surface (i.e. treated with oxygen plasma, APTES, paraformaldehyde, and gelatin) that were stained with the nuclear fluorescence marker DRAQ5, and nuclear stains (white ellipsoids) overlaid with phase contrast images. Separate thresholding of both image channels and subsequent binarization delivers number of cells (nuclei) and image area fraction covered by wrinkles (=contraction in arbitrary units).

FIG. 15 shows fibroblasts grown on wrinkling substrates having an oxidized and cellular adhesion activated surface (i.e. treated with oxygen plasma, APTES, paraformaldehyde) that was provided with fluorescent beads embedded as position markers and coated with gelatin. Isometrically contracting fibroblasts were treated with Cytochalasin D to inhibit contraction. During relaxation, changes in surface wrinkling (phase contrast images) were recorded simultaneously with marker position changes. Phase contrast images were analyzed for wrinkle signal and fluorescent marker displacement was analyzed with traction force microscopy. Heat map diagram shows distribution of forces with white indicating high and black indicating low forces. The substrate deformation calculated from surface marker displacement was correlated with the wrinkling area signal for every change between two image acquisitions. Data shows that wrinkle analysis was linearly related to force analysis with traction force microscopy. Wrinkle number change over cell relaxation was also measured but was not useful as indicator of force amplitude changes.

FIG. 16 shows fibroblasts grown on wrinkling substrates having an oxidized and cellular adhesion activated surface (i.e. treated with plasma, APTES, paraformaldehyde, and gelatin) that were treated with different concentrations of the cell relaxing compound blebbistatin. Wrinkling fractions were quantified over time on the same image fields. Graph 1 demonstrates that the assay and analysis was sufficiently sensitive to quantify relaxation differences between the different treatment groups. Graph 2 was produced from multi-well contraction analysis of a 30 min blebbistatin (50 μM) treated group in comparison with control.

FIG. 17 shows lineage human embryonic stem (hES2) cell-derived cardiomyocytes seeded in different concentrations on biocompatible silicone elastomer films provided with and without APTES/EDAC treatment and matrix protein in different concentrations. (A) Phase contrast images, (B) Quantification of cell covered area. (C) The average number of beating colonies per well and (D, E) the percentage of beating colonies creating wrinkles was quantified for fibronectin (FN 2 μg/ml) and gelatin (2 and 20 μg/ml)-coated wrinkling substrates. (E). To determine the optimal cell concentration for cardiomyocyte wrinkling, cells were seeded at 50,000, 25,000, 10,000 and 5,000 cells/cm² onto APTES/EDAC treated substrates and percentage of beating colonies creating wrinkles was quantified.

FIG. 18 shows hES2-derived cardiomyocytes that were either cultured (A) in the wells of a device similar to that shown in FIG. 12, including a biocompatible silicone elastomer film having a surface that was oxidized with plasma oxidation and activated for cellular adhesion with APTES/EDAC and gelatin; or (B) control culture plastic supports that were also oxidized with plasma oxidation and activated for cellular adhesion with APTES/EDAC and gelatin. In contrast to the wrinkling elastomer, plastic culture did not deform under cell contraction and shape changes of attaching cells were minimal. In the absence of wrinkles, changing cell shape was the only feature that could be analyzed in images. Morphological analysis by thresholding, binarization, and area measurements of bright features (used in wrinkling analysis and commercial imaging systems to quantify cardiomyocyte beating) demonstrated dramatic contraction signal amplification on wrinkling substrates.

FIG. 19 shows cardiomyocyte colonies that were in close vicinity but physically separate. The colonies were analyzed for wrinkle formation (contraction) using a device similar to that shown in FIG. 12 (one well), including a biocompatible silicone elastomer film having a surface that was oxidized with plasma oxidation and activated for cellular adhesion with APTES/EDAC and gelatin. Beating frequency per colony was extracted using Fast Fourier analysis and compared.

FIG. 20 shows that periodic contraction of cardiomyocyte-differentiated hES2 (region of interest 1) and isometrically contracting fibroblast-like hES2s (region of interest 2) are clearly distinct in region-specific contraction analysis. The cells were assessed using a device similar to that shown in FIG. 12, including a biocompatible silicone elastomer film having a surface that was oxidized with plasma oxidation and activated for cellular adhesion with APTES/EDAC and gelatin.

FIG. 21 shows cardiomyocyte-differentiated hES2s that were seeded onto wrinkling biocompatible silicone elastomer films having a surface that was oxidized with plasma oxidation and activated for cellular adhesion with APTES/EDAC and grown to confluence. Surface wrinkling was assessed with different transmission light microscopy contrasting methods, including phase contrast microscopy, dark field microscopy, and differential interference contrast (DIC) microscopy. The last example demonstrates immunofluorescence imaging of cardiomyocytes wrinkling fluorescently labelled gelatin-coated surfaces.

FIG. 22 shows fibroblasts that were seeded onto wrinkling biocompatible silicone elastomer films having a surface that was oxidized with plasma oxidation and activated for cellular adhesion with APTES/EDAC and provided with a layer of fluorescently labelled fibronectin. The fluorescent signal was amplified by the formation of wrinkles and provides a cleaner signal after image binarization due to the fact that cell structures were not labelled.

FIG. 23 shows fibroblasts that were seeded onto wrinkling biocompatible silicone elastomer films having a surface that was oxidized with plasma oxidation and fluorescence-functionalized in sequential steps of treatment with plasma, APTES, and Rhodamine-B-Isothiocyanate, followed by treatment for cell adhesion with fibronectin. The wrinkle signal was visualized in phase contrast transmission light microscopy and in epifluorescence microscopy detecting the Rhodamine signal. Rhodamine functionalization allows detection of wrinkles in the fluorescence channel and eliminates the cell-derived background signals occurring in light microscopy.

FIG. 24(A) shows the wrinkling-derived periodic signal of contracting cardiomyocytes overlaid experimentally with periodic noise. FIG. 23(B) shows Fast Fourier filtering that was used to determine the main frequencies (peaks), and band-pass filtering that was applied to eliminate high frequency peaks (arrows). FIG. 24(C) shows that the filtered signal did not contain the high frequency domain. The assessment was done using a device similar to that shown in FIG. 12, including a biocompatible silicone elastomer film having a surface that was oxidized with plasma oxidation and activated for cellular adhesion with APTES/EDAC and gelatin.

FIG. 25 shows hES2-derived cardiomyocytes that were grown on a biocompatible silicone elastomer film having a surface that was oxidized with plasma oxidation and activated for cellular adhesion with APTES/EDAC and gelatin, and control culture plastic supports that were also oxidized with plasma oxidation and activated for cellular adhesion with APTES/EDAC and gelatin. After 2 weeks, the surface area covered by periodically beating cell masses was quantified.

FIG. 26 shows hES2-derived cardiomyocytes that were grown on a biocompatible silicone elastomer film of 5,000 Pa elastic modulus having a surface that was oxidized with plasma oxidation and activated for cellular adhesion with APTES/EDAC and gelatin. In case 1, cells were immunostained after 7 days of culture for vimentin which is a marker for fibroblastic cells that also develop in these heterogeneous cell populations and for desmin which is a muscle marker. In case 2, cells were immunostained after 7 days of culture for the cardiomyocyte marker α-sarcomeric actinin and nuclei (DAPI).

FIG. 27 shows hES2-derived cardiomyocytes that were grown on a biocompatible silicone elastomer film of different elastic moduli: 5,000 Pa, 10,000 Pa, 15,000 Pa, and 20,000 Pa having a surface that was oxidized with plasma oxidation and activated for cellular adhesion with APTES/EDAC and gelatin. Cells were immunostained after 7 days of culture for the cardiomyocyte marker α-sarcomeric actinin and nuclei (DAPI).

FIG. 28 shows hES2-derived cardiomyocytes that were grown on a biocompatible silicone elastomer film having a surface that was oxidized with plasma oxidation and activated for cellular adhesion with APTES/EDAC and gelatin. Periodically contracting colonies were recorded and live treated with cardiomyocyte affecting drugs in three concentrations (high, medium, low). Ouabain increased beating amplitude, nifidepine decreased contraction amplitude and increased frequency, isoproterenol increased contraction frequency and amplitude, and blebbistatin decreased beating frequency and amplitude to the point of arrest at high concentrations.

### DETAILED DESCRIPTION

Various apparatuses or processes will be described below to provide an example of an embodiment of each claimed invention. No embodiment described below limits any claimed invention and any claimed invention may cover processes or apparatuses that differ from those described below. The claimed inventions are not limited to apparatuses or processes having all of the features of any one apparatus or process described below or to features common to multiple or all of the apparatuses described below. It is possible that an apparatus or process described below is not an embodiment of any exclusive right granted by issuance of this patent application. Any invention disclosed in an apparatus or process described below and for which an exclusive right is not granted by issuance of this patent application may be the subject matter of another protective instrument, for example, a continuing patent application, and the applicants, inventors or owners do not intend to abandon, disclaim or dedicate to the public any such invention by its disclosure in this document.

Current two-dimensional (2D) or three-dimensional (3D) in vitro cell contraction assays are restricted to specialized laboratories and are generally not compatible with HTS technology (Chen et al., 2004). One efficient approach to measure cell contractions is to analyze deformations generated by exertion of cellular forces on 2D elastic culture substrates. 2D cell contraction analysis has typically involved the use of “wrinkling” silicone substrates, in which a nanometer thick film is polymerized on the surface of a viscous silicone oil, and is then wrinkled by cell produced forces, analogous to a hand wrinkling a sheet of paper (Harris et al., 1980). This assay is qualitative and the wrinkling film easily ruptures, excluding quantification of cell forces and HTS. To overcome these limitations and to measure forces, surface-polymerized silicone oil has become gradually replaced by fully polymerized silicone elastomers (Balaban et al., 2001) or polyacrylamide hydrogels (Pelham et al., 1997) with randomly implanted surface markers (Beningo et al., 2002). The defined elasticity of such deformable substrates allows computational calculation of subcellular forces from the displacement of the markers (Balaban et al., 2001). However, assessment of the cell's contractile state at any given instant is not possible, because the marker position in the relaxed cell state is unknown. Implantation of regularly patterned surface markers with micron resolution using soft lithography has ameliorated this problem, but transfer of micropatterned substrates to high throughput screening (HTS) is cost-prohibitive. Deriving cell forces from marker displacements also requires high-resolution microscopy that is currently not compatible with HTS.

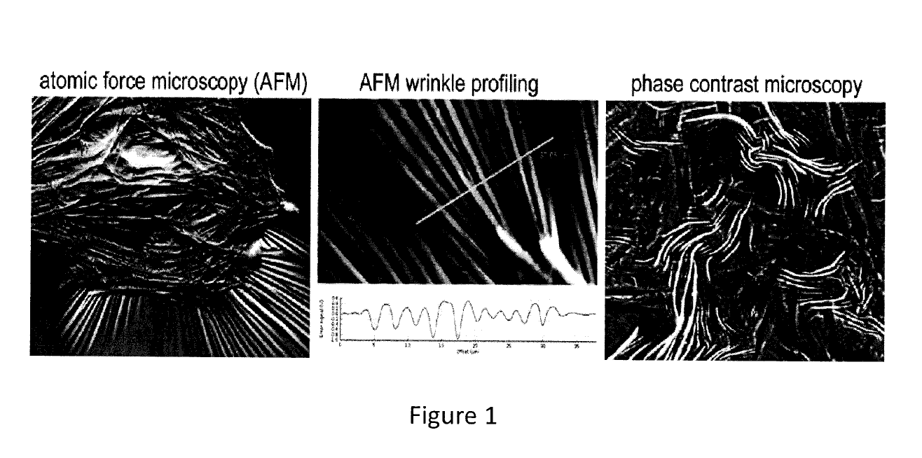

The present application discloses a biocompatible silicone elastomer film that has an oxidized and cellular adhesion activated surface. When cells are adhered to the film, the film allows the cells to contract, and wrinkles when the cells contract, as shown in FIG. 1. The wrinkles can then be analyzed to assess cell contraction. The present application also discloses a device incorporating the biocompatible silicone elastomer film, and methods for assessing cell contraction using the biocompatible silicone elastomer film. The device and methods may be used for high throughput screening.

As used herein, the term “biocompatible” may be used to describe any material that is not substantially harmful and/or not substantially toxic for mammalian cells and living tissues.

The devices and methods disclosed herein may allow for measurement of contractile force. For example, the devices and methods disclosed herein may allow for assessment of force amplitude and frequency of contraction of single cells, such as cardiomyocytes, and cell populations in real-time without cell manipulation (e.g., staining). The devices and methods disclosed herein may also allow for identification of single beating cells, and may allow for quantification of the percentage of synchronously beating cells per population. This may be useful because hES2-derived cardiomyocyte cultures can contain a mixture of cells with distinct nodal/pacemaker, atrial, and ventricular contraction properties.

The devices and methods disclosed herein may also provide a biomimetic mechanical environment for cells, such as cardiomyocytes, by offering a growth surface that matches the physiological stiffness of the heart muscle.

The devices and methods disclosed herein may also be used to assess contractions of other cell types, such as long lasting contractions of non-muscle fibroblasts, as described in detail below.

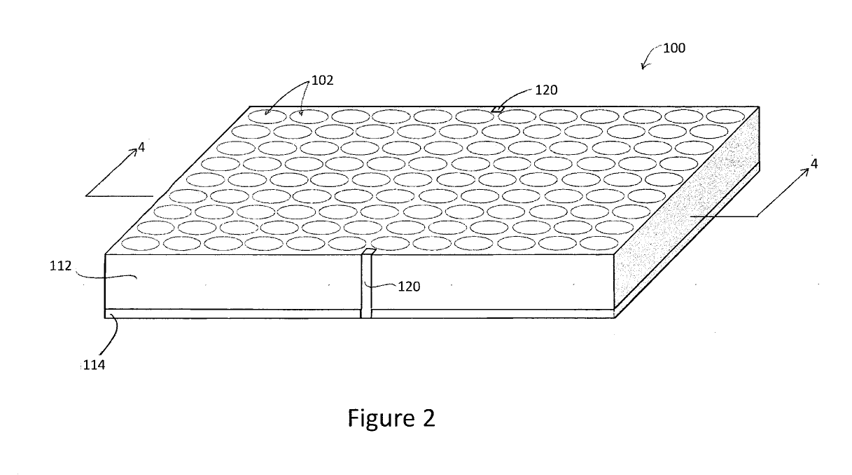

Referring now to FIG. 2, an example device for assessment of cellular contraction is shown. The device may include at least one well. In the example shown, the device is a plate 100 having a plurality of wells 102 (only some of the wells are labeled in the Figures). In the example shown, the device includes 96 wells 102. In alternate examples, the device may include another number of wells, such as 384 wells. Devices including 96 wells or 384 wells may be compatible with pre-existing HTS platforms.

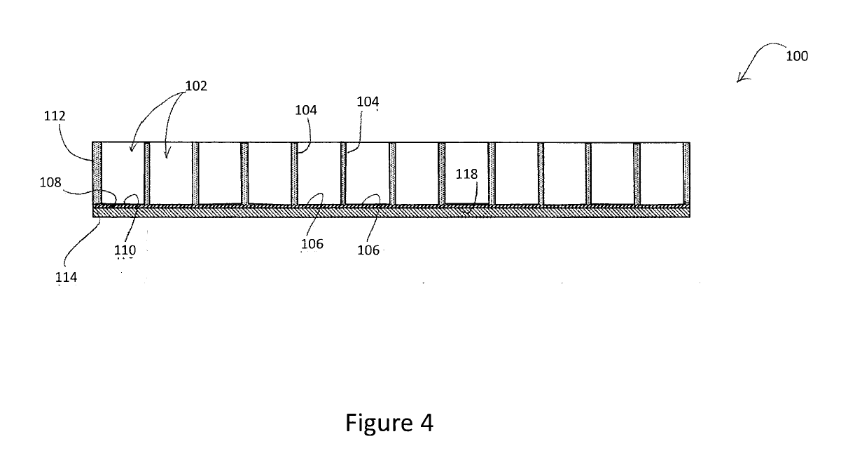

Referring now to FIG. 4, each well 102 includes a well sidewall 104, and a planar well bottom 106 (only some of the sidewalls and well bottoms are labeled). The well bottoms 106 include a coating of a biocompatible silicone elastomer film 108 having an oxidized and cellular adhesion activated surface 110. The biocompatible silicone elastomer film 108 having the oxidized and cellular adhesion activated surface 100 may also be referred to herein as film 108, or biocompatible silicone elastomer film 108. In use, as will be described in further detail below, contractile cells may be adhered to the oxidized and cellular adhesion activated surfaces 110. The biocompatible silicone elastomer film 108 may allow the cells to contract, and may wrinkle when the cells contract. The wrinkles may be analyzed to assess cell contraction.

In the example shown, the wells 102 are generally circular in transverse section, and therefore include only one wall portion forming the well sidewall 104. In other examples, the wells may be another shape in transverse section. For example, the wells may be square in transverse section, and may include four wall portions forming the well sidewall.

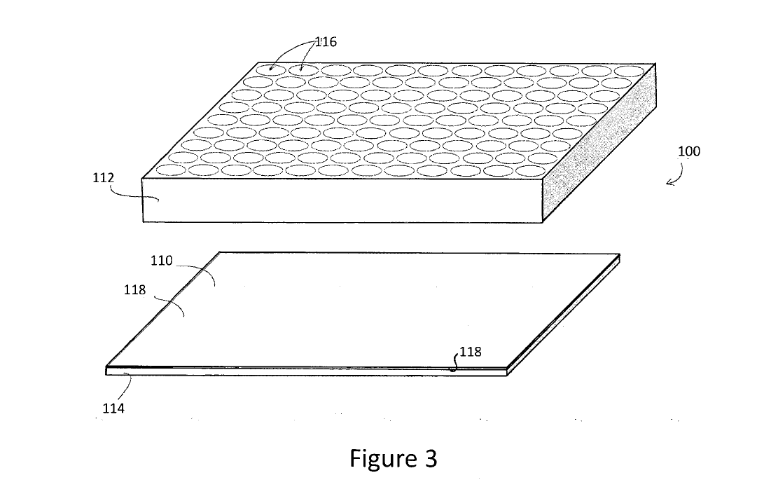

Referring to FIG. 3, in the example shown, the plate 100 is fabricated from two separate pieces, namely an upper plate 112 and a base plate 114.

The upper plate 112 includes a plurality of bottomless wells 116, which define the well sidewalls 104. The upper plate may in some examples be fabricated from polystyrene.

The base plate 114 is formed separately from the upper plate 112 and is secured to the upper plate 112. The base plate 114 includes a planar face 118 that is coated with the biocompatible silicone elastomer film 108 having the oxidized and cellular adhesion activated surface 110. The base plate 114 and film 108 form the well bottoms.

The base plate 114 may be secured to the upper plate 112 by a variety of methods. In the example shown, the base plate 114 is clamped to the upper plate 112 with clamps 120. In alternative examples, the base plate may be adhered to the upper plate, screwed to the base plate, or secured in any other suitable fashion.

The base plate 114 may be transparent, so that in use, the contents of the wells may be viewed through the base plate 114 (e.g. via inverted imaging techniques). For example, the base plate 114 may be fabricated from transparent glass or plastic such as plastic suitable for use in tissue culture, and may have a thickness of between 100 microns and 200 microns. In one specific example, the base plate 114 may have a thickness of about 150 microns.

As noted above, the well bottoms 106 include a coating of a biocompatible silicone elastomer film 108 having an oxidized and cellular adhesion activated surface 110. In some examples, this may be achieved by coating the planar face 118 of the base plate 114 with the biocompatible silicone elastomer (the biocompatible silicone elastomer, prior to oxidation of the surface and activation of the surface for adhesion, may also be referred to as a ‘raw biocompatible silicone elastomer’), followed by oxidizing the surface of the raw biocompatible silicone elastomer film, and activating the oxidized surface of the biocompatible silicone elastomer film for adhesion, to yield the oxidized and cellular adhesion activated surface 110. The coated base plate 114 may then be assembled to the upper plate 112.

By providing a base plate 114 with a generally planar face 118, and coating the base plate 114 with the raw biocompatible silicone elastomer prior to assembling the base plate 114 to the upper plate 114, the wells 102 may generally be provided with a biocompatible silicone elastomer film that has an essentially uniform thickness across all wells 102, and within each well 102.

The base plate 114 may be coated with the raw biocompatible silicone elastomer in any suitable fashion. In some examples, the base plate 114 may be coated with the raw biocompatible silicone elastomer by spin-casting, and may be coated to yield a film thickness of less than 200 microns. For example the film thickness may be between 20 and 40 microns, and more specifically about 30 microns. In some examples, the film thickness may be selected by adjusting the rotation speed of the spin-casting process.

The raw biocompatible silicone elastomer may be, for example, a polydimethylsiloxane (PDMS). For example, the raw biocompatible silicone elastomer may be a polydimethylsiloxane (PDMS) sold under the trade name Sylgard 184® (Dow Corning), Alpagel K (Alpine Technische Produkte GmbH), or Nusil Shore 00 (Silicone Solutions).

In some examples, the raw biocompatible silicone elastomer may be fully polymerized.

As noted above, the biocompatible silicone elastomer film 108 has an oxidized and cellular adhesion activated surface 110.

The surface of the raw biocompatible silicone elastomer film may be oxidized by a variety of methods. In some examples, the surface may be oxidized by plasma oxidation. In other examples, the surface may be chemically oxidized, for example by treatment with hydrogen peroxide and sulfuric acid (Piranha Solution).

The oxidized surface of the biocompatible silicone elastomer film may be activated for adhesion by a variety of methods. In some examples, the oxidized surface may be activated for adhesion by treating the surface with an extracellular matrix (ECM) protein. In further examples, the oxidized surface may be activated for adhesion by silanizing the surface, and treating the surface with ECM proteins.

The oxidized surface may be silanized by a variety of methods. In one example, the surface may be silanized by treating the surface with 3-aminopropyltriethoxysilane (APTES), followed by treatment with either or both of paraformaldehyde and 1-ethyl-3-(3-dimethylaminopropyl) carbodiimide (EDAC).

The ECM protein may include, for example, gelatin, collagen (any type), fibronectin, vitronectin, pronectin, DOPA, N-acetyl glucosamine, BSA, laminin, RGD peptides and derivatives, such as cyclic RGD peptides, and combinations thereof.

In some examples, a fluorescent dye may be incorporated into the biocompatible silicone elastomer film 108. For example, the ECM protein may be fluorescently labeled.

In an alternative example the surface may be provided with a fluorescent layer to facilitate automated analysis. After a sequence of silicone plasma oxygenation and treatment with APTES, amine groups may become available on the silicone surface to react with isothiocyanate (ITC)-functionalized Rhodamine (Rh-ITC). After the reaction, free carboxyl groups of the Rhodamine may react with amine groups of ECM proteins that are added to enhance cell adhesion. Cell attachment and viability are not believed to be affected by adding the Rh-ITC layer. The fluorescence intensity produced by Rh-ITC functionalized wrinkling substrates may be sufficiently strong to detect fluorescent wrinkles with low resolution optics (20× air objective) and short camera exposure times (20 ms).

In an alternative example, in order to activate the surface for adhesion, cell adhesive peptides may be coupled to a bioactive fluorinated surface modifier (BFSM) and blended into the biocompatible silicone elastomer. For example, a known NH2-GK*GRGD-CONH2 (SEQ ID NO: 1) peptide sequence (RGD) with a dansyl label (*) on the lysine residue may be linked via the N-terminal to a BFSM precursor molecule (Ernsting et al, 2005). Fluorinated oligomers, when blended into polymers (before the polymers are coated on surfaces), have been shown to migrate to the surface and generate an interface that promotes cell adhesion. This type of surface modification may enable the introduction of bioactive agents onto the surface in one manufacturing step.

In some examples, a position marker may be embedded in the biocompatible silicone elastomer film 108 to enhance detectability of wrinkles. Position markers can be fluorescent polystyrene or glass beads with diameters ranging from 0.1 to 1 μm that are mixed into the bulk biosilicone elastomer film before spreading on a surface. Position markers can be fluorescent polystyrene or glass beads with diameters ranging from 0.1 to 1 μm that are covalently linked to the elastomer surface after spreading and polymerization on a surface. Position markers can be fluorescent epoxy polymers that are applied to the elastomer surface after spreading and polymerization using photolithography.

In some examples, the stiffness (also referred to as Young's elastic modulus E) of the biocompatible silicone elastomer film 108 may be tuned depending on the type of cell being assessed. For example, cardiomyocytes may produce measurable wrinkles in a biocompatible silicone elastomer film that has a modulus of elasticity of between 500 Pa and 25,000 Pa, more specifically a modulus of elasticity of about 5,000 Pa. Furthermore, fibroblasts may produce measurable wrinkles in a biocompatible silicone elastomer film that has a modulus of elasticity of about 1,500 to 3,000 Pa.

In some examples, in order to view the contents of the wells 102 through the base plate 114, the biocompatible silicone elastomer film 108 may be transparent.

As noted above, the present disclosure also provides a method for assessing cell contraction. The method may be carried out using the device described above, or may be carried out using another device.

The method for assessing cell contraction may generally include adhering contractile cells to an oxidized and cellular adhesion activated surface of a biocompatible silicone elastomer film, as described above. The biocompatible silicone elastomer film may allow the cells to contract and may wrinkle when the cells contract.

The method may further include analyzing wrinkles in the biocompatible silicone elastomer film formed by contraction of the contractile cells. For example, the wrinkles may be analyzed by obtaining an image of the biocompatible silicone elastomer film. The wrinkles may be imaged, for example, by phase contrast microscopy, or atomic force microscopy. Alternatively, in examples wherein the film includes a fluorescent dye, the wrinkles may be imaged by fluorescence microscopy. A proportion of the image that contains the wrinkles may then be determined, and the proportion may be compared to a control. The control may be, for example, a positive or negative control, a reference standard, or the absence or presence of a compound. In some examples, the wrinkles may be analyzed via live imaging, in real time.

In some examples, the wrinkles may be analyzed to quantify a percentage of the cells that are beating or to determine a beating rate of the cells (e.g. where the cells are cardiomyocytes), or to determine a contractile force of the cells.

In some examples, prior to assessing the wrinkles, the cells may be treated to either induce contraction, or inhibit contraction.

In some examples, the method may include assessing the effect of a test compound on cell contraction. For example, the method may include treating the cells with a test compound, and analyzing the wrinkles to assess the effect of the test compound on cell contraction. This may be useful for drug screening. For example, after the wrinkles are analyzed, the test compound may be identified as either an inducer of contraction or an inhibitor of contraction. If the test compound is identified as an inducer of contraction, the test compound may be selected as a candidate treatment for chronic wound healing, low vascular tone, and/or arrhythmia. If the test compound is identified as an inhibitor of contraction, the test compound may be selected as a candidate treatment for at least one of fibrocontractive disease, and/or cancer. If the test compound is identified as an inhibitor of contraction, the method may also comprise selecting the test compound as a candidate smooth and skeletal muscle relaxant.

In some examples, the method may be used to augment cell therapies. For example, the method may be used to identify highly contractile cells (e.g. forces of >3 μN) within a population of low contractile cells (e.g. forces of <3 μN). The highly contractile cells may then be selected for purposes of autologous cell selection for cell therapies. The selected cells may be transplanted into a patient for cell therapy.

The contractile cells may include, for example, fibroblasts, myofibroblasts, epithelial cells, endothelial cells, cardiomyocytes, skeletal muscle cells, smooth muscle cells, mesenchymal stem cells, induced pluripotent stem cells, embryonic stem cells, inflammatory cells, cancer cells, immortalized lineage cells, hepatic stellate cells, pericytes, chondrocytes, chondroblasts, osteoblasts, osteoclasts, astrocytes, myoepithelial cells, glial cells, and neuronal cells. In some particular examples, the cells may be cardiomyocytes. In some particular examples, the cells may be fibroblasts.

The contractile cells may in some examples be in the form of a tissue. For example, tissue may include fibrotic tissue, scar tissue, heart muscle tissue, skeletal muscle tissue, smooth muscle tissue, arterial tissue, venous tissue, connective tissue, nervous tissue, liver tissue, kidney tissue, lung tissue, gastrointestinal tissue, cancer tissue, bone marrow tissue, blood tissue, cartilage tissue, bone tissue, gingiva tissue, skin tissue, tendon tissue, fascia tissue, glandular tissue, embryonic tissue, and reproductive tissue. The tissue can be in the form of thin slices (20-200 μm) of organs or organ parts that attach to the film as a quasi-two dimensional contractile layer. The tissue can be in the form of whole functional excised tissue such as a mouse mammary gland that will attach to the film and wrinkle the film when stimulated to eject milk. In one specific example, the contractile tissue may be scar tissue from fibrotic organs. In another specific example, the contractile tissue may be skeletal and cardiac muscle.

While the above description provides examples of one or more processes or apparatuses, it will be appreciated that other processes or apparatuses may be within the scope of the accompanying claims.

### Example 1

### Introduction

In standard culture, most adherent cells develop contractile actin filament bundles that exert forces to the substrate at sites of integrin-containing focal adhesions (FAs) (Cukierman et al., 2002, Geiger et al., 2001). The magnitude of force varies between different cell types which, at least to some extent reflects cell function in the origin tissue; e.g. cultured contractile heart, skeletal and smooth muscle cells exert high forces, whereas epithelial cells and migrating fibroblasts produce comparably low forces. During physiological tissue repair and pathological development of fibrosis (Wynn, 2008, Klingberg et al, 2013), low contractile fibroblasts differentiate into myofibroblasts in response to changes in the chemical and mechanical microenvironment (Hinz, 2010). This transition is characterized by de novo expression of α-smooth muscle actin (α-SMA) whose incorporation into stress fibers renders fibroblastic cells highly contractile (Hinz et al, 2001a, Hinz et al., 2002). α-SMA has been identified as mechano-sensitive protein: releasing myofibroblasts from external stress by growing them on compliant substrates leads to the disassembly of α-SMA from stress-fibers within one day (Goffin et al., 2006). Stress release has previously been shown to reduce subsequent α-SMA protein expression over a period of several days (Arora et al, 1999). However, a detailed time-course analysis correlating development of myofibroblast intracellular tension with changes in α-SMA localization and protein expression upon growth on different compliant substrates has remained elusive because a method that allows simultaneous analysis of these parameters was not yet available.

With the devices and methods disclosed herein, it is demonstrated that myofibroblasts that have been transferred to oxidized and cellular adhesion activated biocompatible 200 μm thick silicone elastomer films initially keep their level of differentiation and contraction and produce wrinkles on films with an elastic modulus of up to ˜21,000 Pa. Wrinkle capacity becomes restricted to highly compliant films of ≧3,000 Pa within 36 hours, correlating with the loss of α-SMA from stress fibers at unaltered protein expression levels. Continued growth on 200 μm thick films with a modulus of below ˜16,000 Pa subsequently lead to dramatic reduction of α-SMA protein expression within a few days. The phenotypic change was suppressed on wrinkling elastomers with a thickness of <50 μm. This finding is consistent with previous findings that cells are able to mechanosense the stiff support material (plastic or glass) underlying very thin elastic polymer substrates (Buxboim et al., 2010).

### Materials & Methods

### Cell Culture and Drugs

Primary rat lung myofibroblasts and subcutaneous fibroblasts (SCF) were derived from explants and cultured from passage 2-7. All cells, including lineage aortic smooth muscle cells (A7r5) and rat embryonic fibroblasts (REF-52) were cultured in DMEM (Gibco-BRL, Basel, CH), containing 10% FCS and antibiotics. Cytochalasin D (Sigma) was used at 1-10 μM, blebbistatin (Calbiochem, Darmstadt, Germany) at 10 μM, Y27632 (Calbiochem) at 10 μM and lysophosphatic acid (LPA) (Sigma) at 10 μM. For FA maturation analysis, REF-52 were stably transfected with β3 integrin-GFP (16), full-length GFP-paxillin (Zamir et al, 2000), and α-SMA-GFP (Clement et al., 2005), using Fugene 6 (Roche, Reinach, CH).

### Production and Surface Treatment of Polydimethylsiloxane (PDMS) Films

Biocompatible silicone elastomer films were produced by mixing PDMS curing agent and base (Sylgard 184, Dow Corning, Midland, Mich.) in ratios between 1:40 and 1:120 (w/w) for 3 h at RT; lower proportions of curing agent resulted in insufficient and non-reproducible polymerization. To reduce pipetting errors and to ameliorate the mixing process, curing agent can be pre-diluted in toluene without changing the elastic properties of the polymerized material. PDMS films of 200 μm thickness were produced by distributing the respective volume with a pipette tip onto glass coverslips (#0, Karl Hecht KG, Sondheim, Germany) at the bottom of homemade observation chambers or on standard culture dishes. Substrates were degassed in a desiccator and polymerized for minimum 3 d at RT. Polymerized films were kept at RT for up to two months without changing compliance. Alternative polymerization protocols using higher curing temperatures are possible but will have an influence on the elastic modulus of the film (Lee et al., 2004).

After sterilization with 70% ethanol, three different protocols of surface treatment were applied: 1) no specific treatment, 2) physico-chemical oxidation with oxygen plasma for 45 sec at 50 W, and 0.3 Torr, using a glow discharge apparatus (Plasmaline 100, Tegal, Petaluma, Calif.) (i.e. plasma oxidation) and 3) chemical oxidation with H₂SO₄ (30%) for 5 min at RT (i.e. sulfuric acid oxidation). In some conditions, the treatments were followed by activation of the surfaces for cellular adhesion, by thorough washing with distilled water, silanization with 2% 3-aminopropyltriethoxysilane (APTES) (Sigma, St Louis, Mo.) in ethanol for 15 min at RT and extensive washing with 100% ethanol. The subsequently dried surface was treated with 0.1% paraformaldehyde (PFA) in PBS for 15 min on a shaker at RT and washed again with distilled water. All surfaces were finally coated with 10 μg/ml collagen type I (Sigma) for 1 h at 37° C.; other extracellular matrix (ECM) proteins such as fibronectin, vitronectin, BSA, and laminin were tested to absorb equally well.

### Determination of the Elastic Modulus of PDMS and Force Approximation

To determine the viscoelastic properties of biocompatible silicone elastomer films with different base-to-curing agent mixing ratios, the dynamic shear modulus was first measured using a high resolution rheometer (CVO 120, Bohlin Instruments, Worcestershire, United Kingdom) (Yeung et al., 2005). PDMS was polymerized into cylindrical-shaped samples with a height of 5 mm to fit between two parallel plates with a diameter of 20 mm. The storage shear modulus was determined from the shear stress in phase with small amplitude oscillatory shear strain that was adapted for each sample in a range of 1-100 mHz. From the shear modulus G′, the Young's elastic modulus E was then calculated by considering a Poisson's ratio υ of 0.5 that is typical for isotropic incompressible solids like rubber: E=G′·2(1+υ). Second, the compliance of PDMS films was assessed after different surface treatment with the use of atomic force microscopy (AFM). PDMS samples similar to those used for cell culture were probed wet with non-functionalized spherical-tipped AFM cantilevers (Novascan Technologies Inc., Ames, Iowa) (spring constant: 60 pN/nm, borosilicate sphere-size: 5 μm), mounted on a XE-120 AFM (PSIA Inc., Santa Barbara, Calif.). Force-indentation curves (n=50) at a rate of 2 μm/s were produced from each sample and the elastic modulus was fitted with a conventional Hertz sphere model (Dimitriadis et al., 2002, Engler et al., 2004b)

To approximate the cell force leading to appearance of wrinkles, wrinkles were experimentally induced by pinching different PDMS films between a displaced deflecting microneedle and a stiff needle, fixed to the substrate (Hinz et al., 2001, Lee et al., 1994). Forces were then averaged from 15 different regions by considering the flexible needle stiffness (nN/μm) and its deflection (μm) at the moment of first wrinkle appearance.

### Antibodies, Microscopy and Image Analysis

To preserve wrinkles for immunostaining, care was taken to keep the samples covered with solution during the whole procedure. After rinsing with serum-free medium, cells were fixed for 10 min at RT by adding 6% ice-cold PFA/PBS to an equal volume of remaining medium. Following permeabilization for 5 min with 0.2% Triton X-100 (TX-100), primary antibodies were applied for 60 min at RT directed against vinculin (hVin-1, mouse IgG1, Sigma) and α-SMA (αSM-1, mouse IgG2a) (Skalli et al, 1986). Secondary antibodies TRITC- and FITC-conjugated goat anti-mouse IgG1 and IgG2a (Southern Biotechnology Associates Inc., Birmingham, Ala.) were applied for 60 min. F-actin was probed with Phalloidin-Alexa 647 (Molecular Probes, Eugene, Oreg.) and DNA with DAPI (Fluka, Buchs, CH). All washing steps and antibody dilutions were performed with 0.02% TX-100 in PBS to reduce buffer surface tension.

Confocal images of fixed specimen were acquired using a 40× oil immersion objective (HC PL APO, NA 1.25-0.75, Leica, Glattbrugg, CH), mounted on an inverted confocal microscope (DM IRE2 with a laser scanning confocal head TCS SP2 AOBS, Leica). Immunofluorescence images were superposed with transmission confocal images that have been processed with Adobe Photoshop to highlight the position of wrinkles. Live videomicroscopy was performed under controlled temperature and CO₂ conditions using a Zeiss Axiovert 200M (Zeiss, Oberkochem, Germany), equipped with a spinning disk Nipkow confocal head (Yokogawa CSU10), Photometrics CoolSNAP-HQ CCD camera and Metamorph 6.0 acquisition software (Visitron Systems, Puchheim, Germany). Phase contrast sequences were taken at a rate of 1 frame/min and live fluorescence images every 30 min using 20× (Plan-Apochromat, Ph2, NA 0.5) and 40× (Plan-Neofluar, Ph3, NA 1.3 oil immersion, Zeiss) objectives. Kymographs were produced from image sequences using MetaMorph (Visitron Systems, München, D) and figures were assembled with Adobe Photoshop (Hinz et al, 1999).

### Western Blot Analysis

Total cell lysates were obtained as previously described with the exception that no cell scraper was used (Hinz et al., 2001a, Hinz et al, 2003). Lysates were run on 10% SDS gels, transferred to nitrocellulose membranes and blotted using the same primary antibodies as in immunofluorescence, HRP-conjugated secondary goat anti-mouse antibody (Jackson ImmunoResearch, West Grove, Pa.) and ECL chemiluminescence detection (Amersham, Rahn, Zurich, CH). The ratio between all digitized band densities of one blot was quantified (ImageQuant V3.3, Molecular Dynamics, Sunnyvale, Calif.) and normalized to housekeeping vimentin (mouse clone V9, DAKO, Glostrup, DK) expression.

### Results

### Contractile Fibroblasts Wrinkle Biocompatible PDMS Elastomer Films

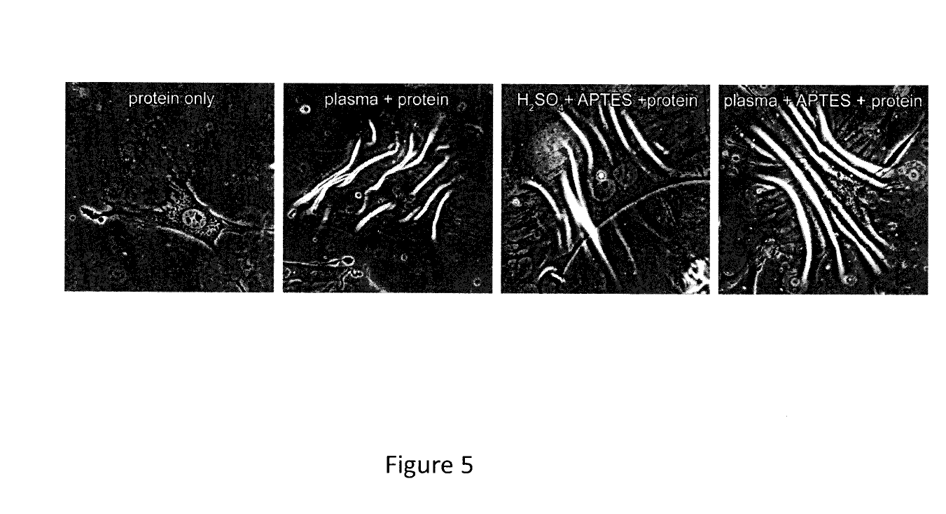

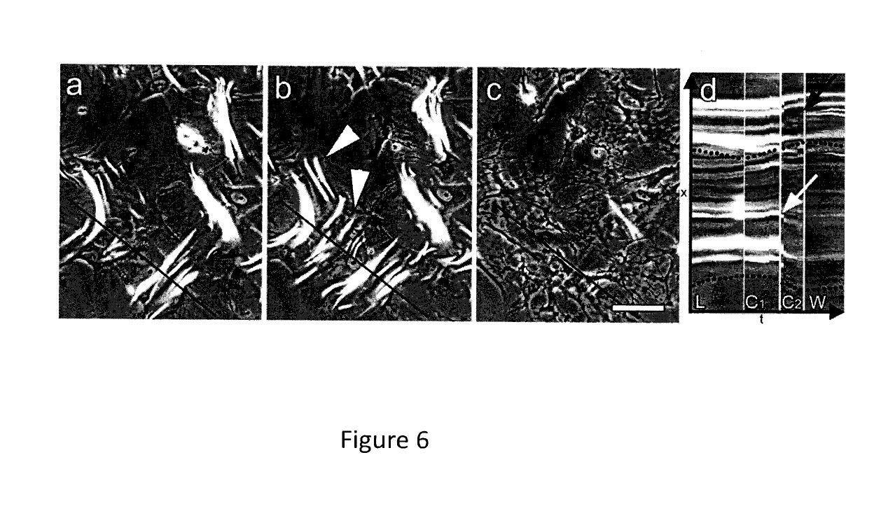

It has previously been reported that contractile cells generate distortions in PDMS elastomer films produced with a curing agent-to-base ratio of 1:50 (Young's modulus of ˜25,000 Pa); however, deformations are in the range of a few microns and are only detectable with the aid of surface markers and high resolution objectives (Balaban et al, 2001, Goffin et al, 2006). It was hypothesized that PDMS films with lower curing agent ratio and consequently higher compliance should be subjected to larger deformations that are visible without position markers. Indeed, fibroblasts produced wrinkles perpendicular to the cell's axis in the surface of 1:100 PDMS films to which ECM proteins have been absorbed after surface oxidation (‘plasma+protein’), as tested for collagen type I (FIG. 5), fibronectin and vitronectin. Absorbing ECM proteins to untreated PDMS films promoted attachment and spreading of fibroblasts; however, cells tended to detach as monolayer sheets when reaching high confluence and did not wrinkle (‘protein only’). Cell detachment at confluence was reduced significantly by oxidizing 1:100 PDMS with oxygen plasma (‘plasma activation’), followed by treatment with ECM proteins (FIG. 5). Finally, covalent binding of ECM proteins to sulfuric acid- or plasma oxidized and APTES-activated PDMS (FIG. 5) (‘H₂SO₄+APTES+protein’ and ‘plasma+APTES+protein’) resulted in cell attachment even in highly confluent culture. On all surfaces, wrinkles were formed and cell morphology was comparable to that on standard culture dishes. Wrinkle formation was dynamic and always reversible (FIG. 6) as here analyzed in detail for one contractile cell using kymograph analysis (Hinz et al., 1999). The number and extension of wrinkles increased after inducing fibroblast contraction with LPA (FIG. 6) and was completely abolished by depolymerizing stress fibers with cytochalasin D (FIG. 6) and inhibition of cell contraction with Y27632. Restoring stress fibers and cell contraction by subsequent washing resulted in the re-formation of wrinkles (FIG. 6).

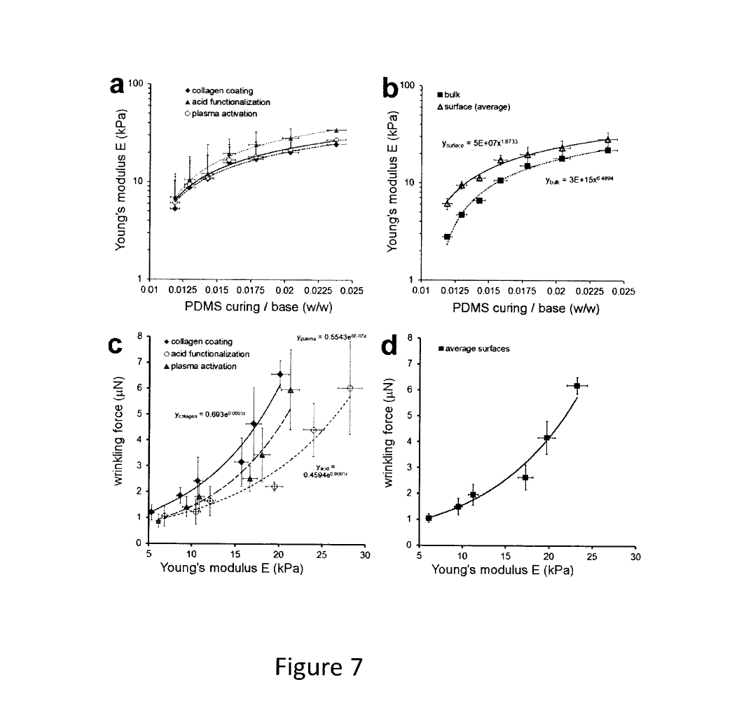

Notably, wrinkle morphology changed with surface treatment: on only collagen-absorbed surfaces, no wrinkles were formed. On surfaces, treated with plasma and subsequent collagen coating, wrinkles were numerous and were mostly restricted to the cell and its close vicinity (FIG. 5). In contrast, wrinkles produced on oxygen plasma-oxidized and chemically oxidized PDMS, followed by APTES and paraformaldehyde treatment were thicker and protruded several tens of microns away from the cell. The propagation of cell-generated wrinkles on the surface of oxygen plasma-oxidized and chemically oxidized PDMS films (FIG. 5) suggested that the functionalization process created a thin surface film that may exhibit higher rigidity than the underlying PDMS. To test this possibility, the Young's (elastic) modulus of non-functionalized PDMS samples (i.e. raw biocompatible silicone elastomer films) was first calculated from their storage shear modulus (FIG. 7a) and surface Young's modulus on the (sub-) cellular level was assessed using AFM (FIG. 7b, c). With decreasing curing agent-to-base ratio from 1:40 to 1:110 the Young's modulus of the raw PDMS decreased from ˜47,000 to 3,400 Pa (FIG. 7a). The relation between mixing ratio R and elastic modulus E was best fitted (r²=0.98) with the exponential expression E(bulk)=46.7e^{0.86R}.

The elastic modulus of the film after collagen absorption was comparable with the raw material, whereas oxygen plasma oxidation significantly increased the substrate stiffness; chemical oxidation moderately increased substrate surface stiffness.

With known elastic modulus, it is possible to predict the minimum cell force leading to wrinkle formation after calibration of the film (Burton et al., 1999, Hinz et al, 2001a). First, biocompatible silicone elastomer films were deformed by moving a flexible microneedle on its surface until first wrinkles occurred; the minimum wrinkling force was calculated from the known needle stiffness (nN/μm) and its deflection (μm). Second, the obtained values were controlled by calculating forces from the displacement of the film surface (i.e. of the needle tip) until first wrinkle formation and its Young's modulus, using previously described mathematical analysis (Goffin et al., 2006). Both approaches produced similar results (FIG. 7c). Third, minimum wrinkling forces were expressed as a function of the substrates' elastic modulus (FIG. 7d).

### PDMS Films are Optimized for Different Cell Types by Tuning Compliance

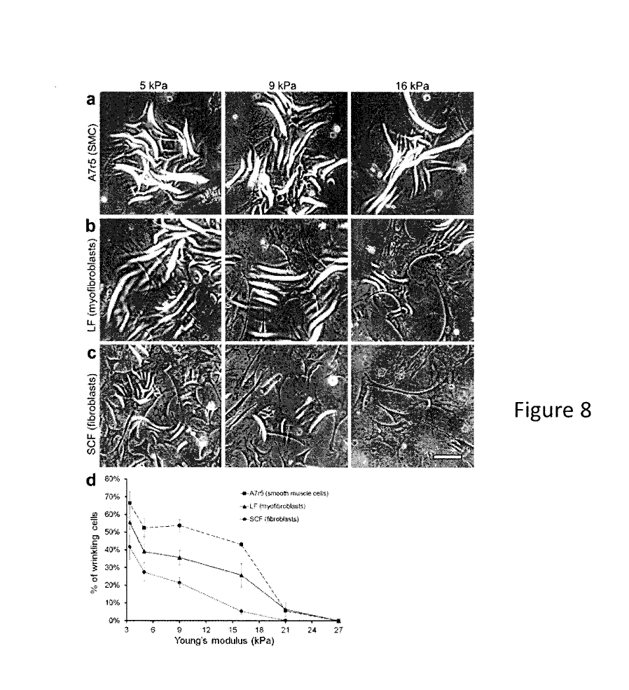

To test the capacity of biocompatible silicone elastomer films to compare the contractile activity of cells, different cell types were grown for 1 d on biocompatible silicone elastomer films having a surface that was oxidized with sulfuric acid and activated for adhesion with APTES silanization and paraformaldehyde cross-linked collagen type I. Rat aorta smooth muscle cells (A7r5) (FIG. 8a, d) and myofibroblasts (LF) (FIG. 8b, d) produced wrinkles in films with a Young's modulus of 59,000 Pa. Smooth muscle cells wrinkled even stiffer films with a modulus of up to 16,000 Pa. In contrast, wrinkle formation by fibroblasts (SCF) was only efficient at an elastic modulus 53,000 Pa (FIG. 8c, d). To further compare the contractile potential of cells, the percentage of wrinkling cells on each film was quantified. In general, highly contractile muscle cells exhibited higher percentages of wrinkling cells compared with myofibroblasts and low contractile fibroblasts at any given film stiffness (FIG. 8d).

### Correlation of Cell Contraction and Protein Expression

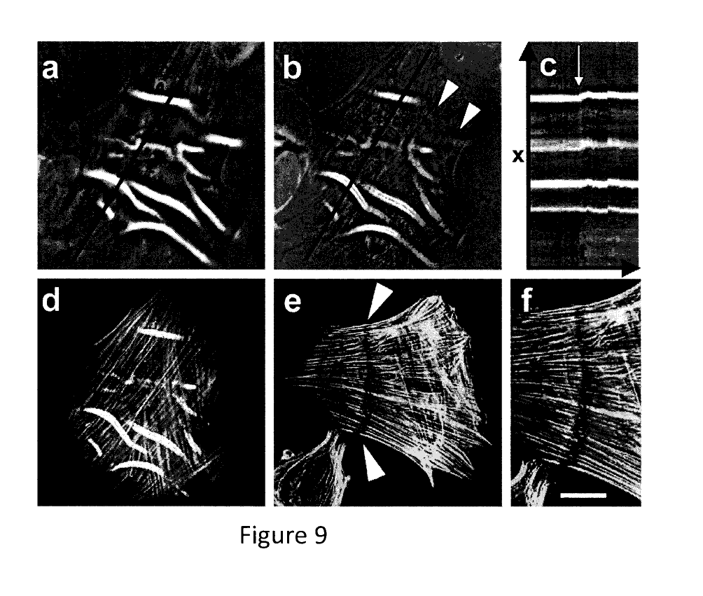

In conditions where only a fraction of cells produces wrinkles (FIG. 8d), it is of interest to determine the molecular basis for this higher contraction compared with non-wrinkling cells, grown on the same substrate. Non-wrinkling deformable substrates are inefficient to correlate contractile activity with protein expression and localization after cell fixation since the non-distorted state of the substrate is generally not known or requires technically demanding micro-structuring (Balaban et al., 2001, Goffin et al., 2006). Here, a standard immunostaining protocol is provided that preserves wrinkles for subsequent microscopic analysis. In this example, biocompatible silicone elastomer films having a surface that was oxidized with sulfuric acid and activated for adhesion with silanization and cross-linked collagen type I were used. During this procedure the number and extension of wrinkles generated by a living cell (FIG. 9a) was almost unaltered and few wrinkles disappeared after fixation with PFA (FIG. 9b, arrowheads, FIG. 9c). This allowed direct correlation between the contractile state of the cell and the expression and localization of proteins like F-actin and α-SMA in still images (FIG. 9d). Due to the elastic nature of the substrate which is not altered by chemical fixation, the tension stored in the wrinkle can literally break stress fibers in 5-10% of the fixed cells (FIG. 9e). Such ‘fractured’ cells should thus be considered when quantifying the percentage of wrinkling cells in a population (see below) even when the corresponding wrinkles may have disappeared.

### Cell Contractile Capacity Adapts to Film Stiffness Over Culture Time

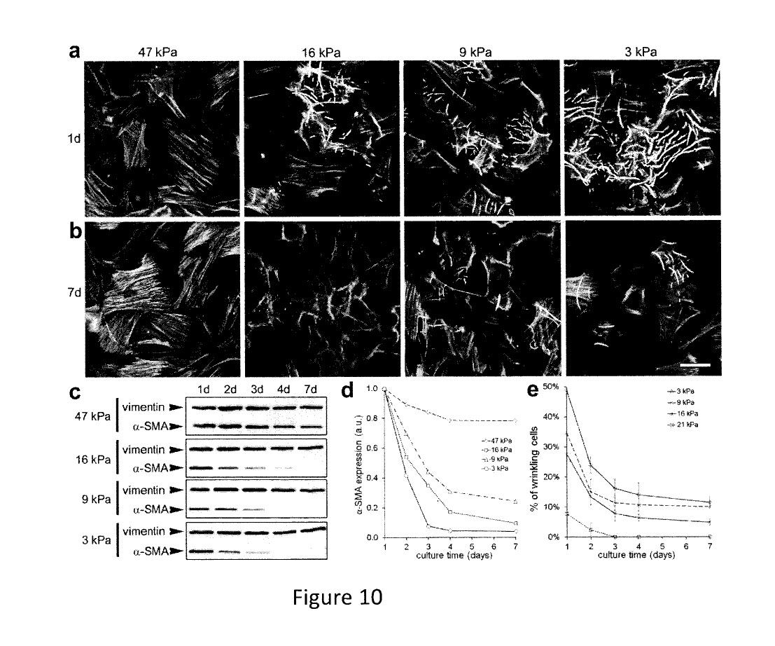

Although this fact is often neglected, the use of elastic substrates, such as wrinkling films, for force analysis is an invasive technique and bears the risk to alter the contractile behavior of the cell which is actually to be measured. It has previously been shown that myofibroblasts lose α-SMA from stress fibers after 12 h growth on PDMS films with an elastic modulus of 516,000 Pa; after this time protein levels are unaltered (Goffin et al., 2006). To further investigate how α-SMA expression levels change together with cell contraction over culture time on soft substrates, LF were cultured for 1-5 d on PDMS films with a modulus of 47,000 Pa, 16,000 kPa, 9,000 Pa, and 3,000 Pa. After 1 d culture on 200 μm thick substrates, α-SMA still localized to stress fibers on all films (FIG. 10a-d) and protein levels remained unchanged compared with stiffer non-wrinkling PDMS (47,000 Pa) (FIG. 10a-d). With prolonged culture, α-SMA protein expression was down-regulated at rates that increased with decreasing film stiffness. Expression of α-SMA was completely lost after 7 d culture on 16,000 Pa, after 5 d on 9,000 Pa and already after 3 d on 3,000 Pa substrates; in contrast, culture on 47,000 Pa stiff substrates preserved the differentiated myofibroblast phenotype (FIG. 10a-d).

Concomitant with the loss of α-SMA, fibroblast contraction was dramatically reduced over culture time on compliant films (FIG. 10e). In addition, this experiment confirms and extends previous findings that only α-SMA-positive myofibroblasts are able to produce wrinkles in films with a compliance of 16,000-9,000 Pa (FIG. 8). Stiffer films are also wrinkled, although less efficiently by fibroblasts exhibiting α-SMA-negative stress fibers, which was evident after 7 d culture (FIG. 10 b, d). To exclude that the diminution of wrinkling cells on soft substrates over time was due to alteration of film stiffness by the culture conditions, 3,000 Pa substrates were re-used after 7 d culture. Myofibroblasts that lost contraction were removed by trypsinization and replaced by fresh α-SMA-positive LF, which again developed strong wrinkle formation after 1 d culture. Hence, to assess the contractile capacity of cells with 200 μm-thick deformable films in general, and with the biocompatible silicone elastomer film of the disclosure in particular, culture time may be limited to approximately 1 d when working with 200 μm thick films. In the following example the layer thickness was reduced to below 30 μm to eliminate a phenotype changing influence of the soft substrate on cells.

### Example 2

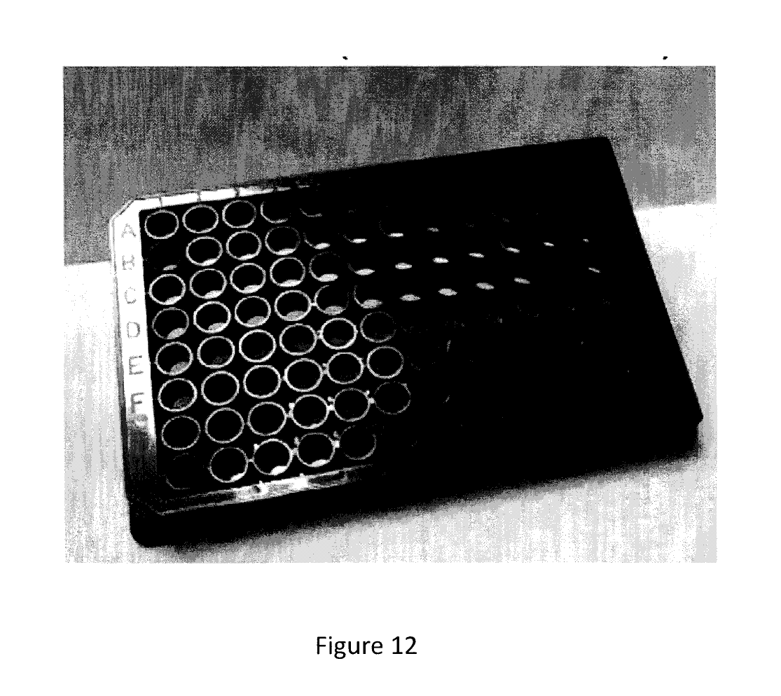

A device for assessing cell contraction was prepared, and is shown in FIG. 12. The device was assembled from a bottom-less multi-well cast (also referred to as an ‘upper plate’) and a 150 μm thick custom-made glass support (also referred to as a ‘base plate’), provided with a 30 μm thick layer of biocompatible silicone elastomer in a spin-casting process. This procedure, rather than distributing the polymer well-by-well, provided even thickness of the PDMS layer across the whole device with micron-precision.

The following biocompatible silicone elastomers were successfully tested in wrinkling applications: polydimethylsiloxane (PDMS, Sylgard 184, Dow Corning), Alpagel K (Alpina Technische Produkte GmbH) and Nusil Shore 00 (Silicone Solutions).

It is conceivable that wrinkling can be obtained with other biocompatible silicone elastomers. Such other elastomers may include, but are not limited to, biocompatible silicone elastomers that are fully polymerized with an elastic modulus between 2,000-10,000 Pa.

Various surface treatments were applied to the biocompatible silicone elastomer film, as described below in Example 3.

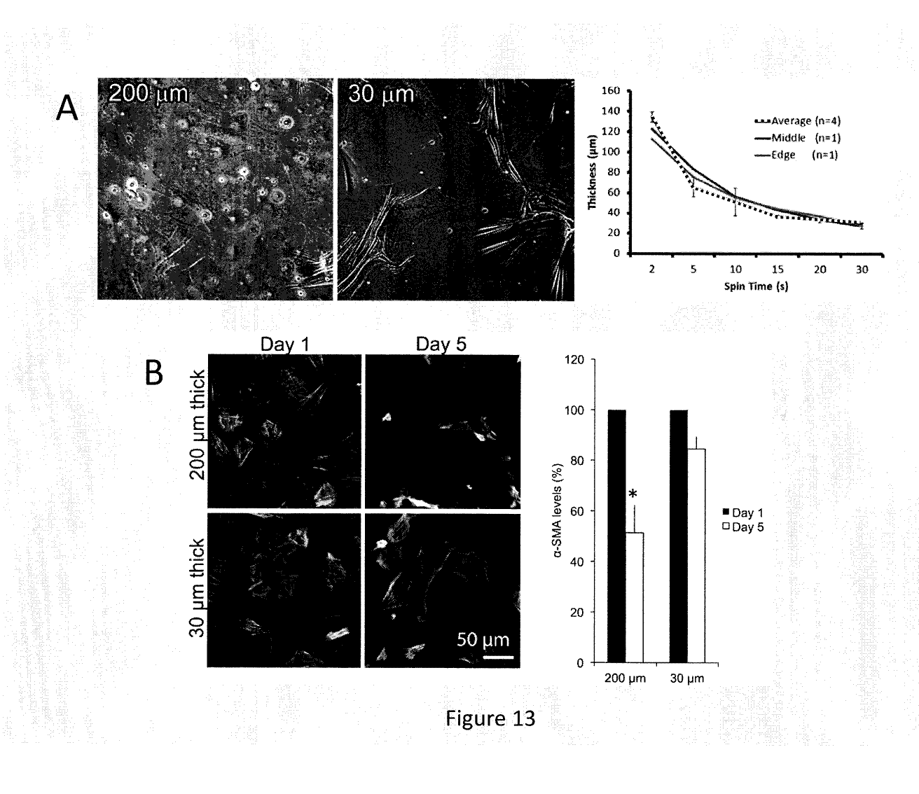

The thickness of the biocompatible silicone elastomer (Alpagel) was reduced from 200 μm to 30 μm by improving the spin-casting process. Thinner layers wrinkle more efficiently and provide dramatically improved optical qualities for automated analysis (FIG. 13A). Because the device may be used with inverted imaging methods, observing cells through the silicone and glass base plate, thinner layers produce better signal-to-noise ratios and accommodate the shorter working distances of high resolution objectives.

FIG. 13A shows fibroblasts induced wrinkles in biocompatible silicone elastomer films formed by spin-casting having an oxidized and cellular adhesion activated surface (plasma, APTES, paraformaldehyde, gelatin). The films have a film thickness of 200 microns, and 30 microns, respectively. Thickness measurements performed at the edges and in the center of the film demonstrated even thickness across the whole surface. FIG. 13B demonstrates another beneficial effect of using thin layers of biocompatible silicone elastomer films, which is reducing the effect of substrate compliance on cell types that require a stiff environment to develop contractile features. Accordingly, soft (5,000 Pa) thin (30 μm) biocompatible silicone elastomers preserve contractile fibroblast features (expression of α-SMA in stress fibers), which are lost over time in soft (5,000 Pa) thick biocompatible silicone elastomers (200 μm).

### Example 3

A device similar to that shown in FIG. 12 was used to grow rat lung fibroblasts. The cells were grown for 1 day on biocompatible silicone elastomer films (PDMS) that were subjected to different surface treatments to improve wrinkle morphology and cell adhesion (FIG. 5). In FIG. 5, “Protein” indicates treating the surface with ECM proteins; “plasma” indicates that the surface was oxidized via plasma oxidation, “APTES” indicates that the surface was treated with APTES followed by treatment with paraformaldehyde, and “H₂SO₄” indicates that the surface was treated with sulfuric acid.

It was determined that plasma oxygenation alone provided only poor cell adhesion to biocompatible silicone elastomer films, and surface coating with cell-adhesive moieties (e.g., ECM proteins) was required to permit sufficient force transmission for substrate wrinkling.

As seen in FIG. 5, oxidizing the surface of the biocompatible silicone elastomer and activating the surface for adhesion yields excellent cell-adhesiveness and substantially amplifies the wrinkling properties of the film.

### Example 4

With a known elastic modulus of the biocompatible silicone elastomer film, it is possible to relate the formation of wrinkles to the minimal force exerted by cells. However, the assay is in essence semi-quantitative and optimized to quantify relative changes in cell contraction (increase/decrease of wrinkles) or to compare different experimental conditions or cells (more/less wrinkles). By embedding position markers in the biocompatible silicone elastomer film in select experiments, a linear relationship was demonstrated between wrinkle signal and cell force. In other words, if cell force doubled, the wrinkling signal was increased 2-fold. For HTS wrinkling analysis, a straight-forward protocol was established to automatically quantify wrinkling intensity (relative contractile force). The fact that wrinkles generate a bright signal in transmission white light microscopy that is intensified by phase contrasting methods was exploited.

Using a thresholding function with subsequent image binarization and feature detection criteria to exclude circular cell signals, linear wrinkle signals were selectively retained. Dividing the area covered by wrinkles by the total image area delivers the wrinkle fraction and is expressed as contraction (arbitrary units).

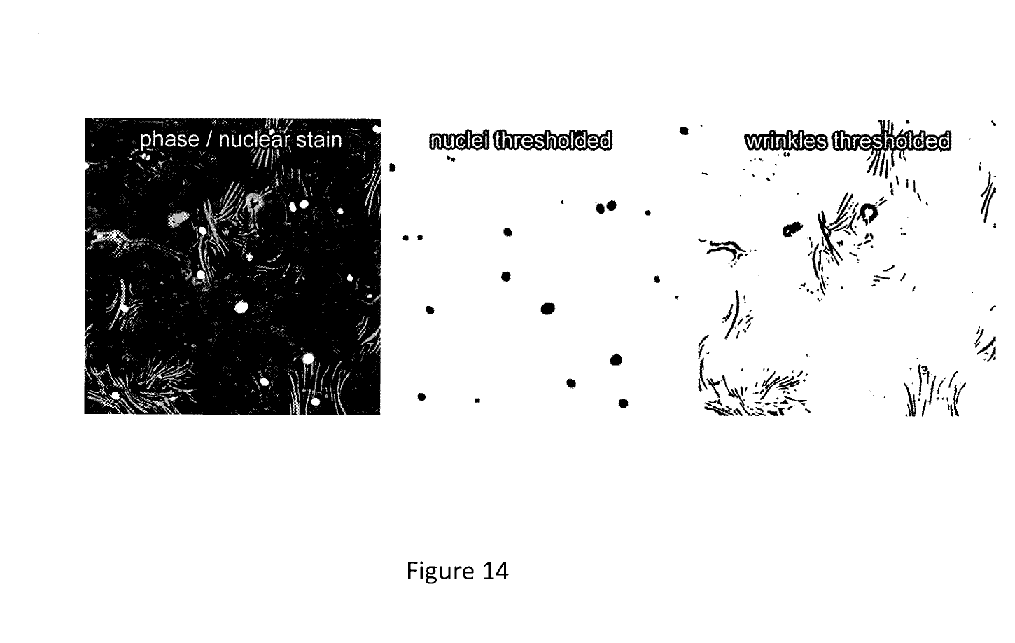

For comparison of different experimental conditions, wrinkle analysis may be combined with a nuclear stain to normalize for cell number in the image field. Referring to FIG. 14, fibroblasts were grown on biocompatible silicone elastomer films having an oxidized and cellular adhesion activated surface (plasma, APTES, paraformaldehyde, gelatin). The fibroblasts were stained with the nuclear fluorescence marker DRQ5 and nuclear stains (white ellipsoids) were overlaid with phase contrast images. Separate thresholding of both image channels and subsequent binarization delivers number of cells (nuclei) and image area fraction covered by wrinkles (=contraction in arbitrary units).

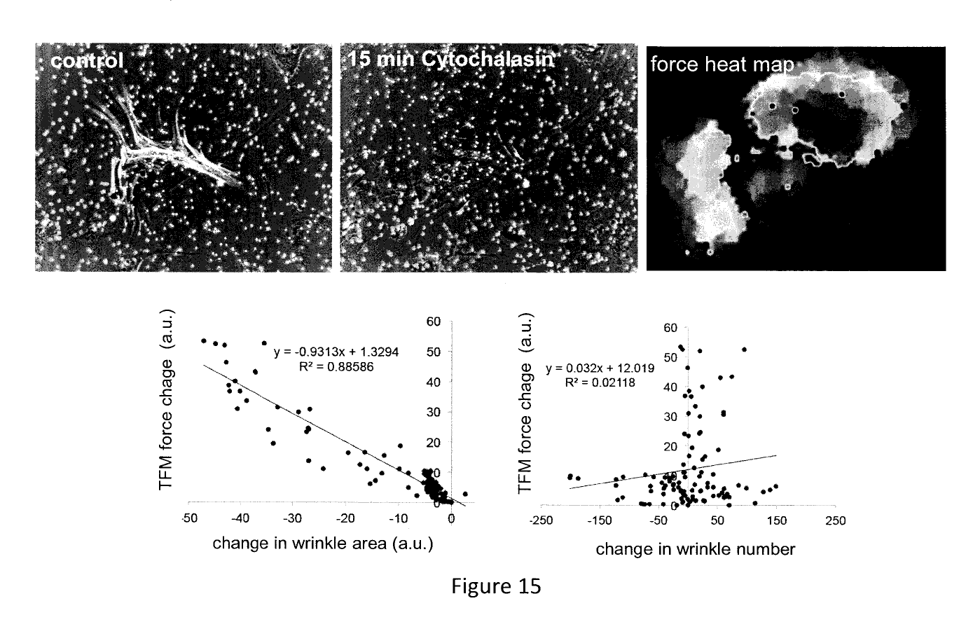

To establish the relationship between wrinkle signal and cell force, fluorescent beads were embedded as position markers in the wrinkling silicone surface. Referring to FIG. 15, marker positions were simultaneously recorded with changes in surface wrinkling upon chemical relaxation of contracting fibroblasts. Phase contrast sequences were analyzed for wrinkle signal according to our standard thresholding procedure and traction force microscopy was used to calculate force fields from the displacement of surface markers. Traction force microscopy is the most widely used benchmark technology to calculate forces developed by attached cells but requires high-resolution optics (40×) and respectively small image fields. Traction force microscopy has ˜100-times higher demands on computing time and data storage than wrinkle analysis and requires high resolution optics. Experiments with fibroblasts demonstrate a linear relationship between changes in wrinkle signal and force over the course of relaxation induced by the toxin Cytochalasin as shown in FIG. 15. FIG. 15 additionally shows that wrinkle number may not be a suitable parameter to correlate with force. These results are predicted to be confirmed with cardiomyocytes since wrinkle/force relation exclusively depends on substrate mechanics and not on cell behavior.

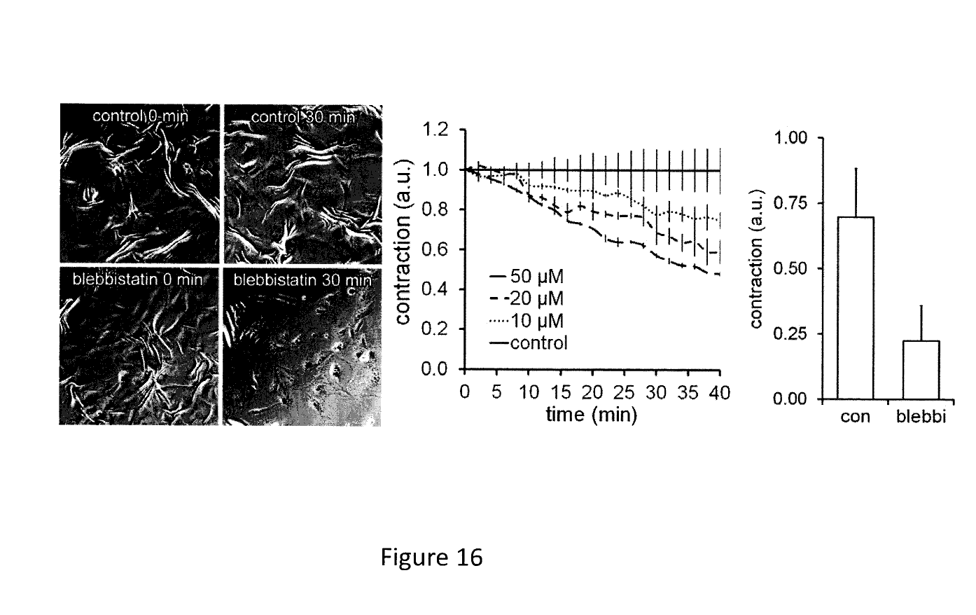

Referring to FIG. 16, normalization is not required when comparing the same image fields before and after treating cells with contraction-inducing or -reducing compounds, as is shown in FIG. 16 for the relaxing drug blebbistatin on lung fibroblasts. Fibroblasts were grown on elastomer film having an oxidized and cellular adhesion activated surface (plasma, APTES, paraformaldehyde, gelatin) and treated with different concentrations of the cell relaxing compound blebbistatin and wrinkling fractions were quantified over time on the same image fields. Graph 1 demonstrates that the assay and analysis was sufficiently sensitive to quantify relaxation differences between the different treatment groups. Graph 2 was produced from multi-well contraction analysis of a 30 min blebbistatin (50 μM) treated group in comparison with control.

### Example 5

Forces developed by cardiomyocytes are 10-50-times higher than those developed by non-muscular fibroblastic cells. Consequently, the stiffness of the biocompatible silicone elastomer (i.e., the sensitivity of the force sensor) may be tuned for use with cardiomyocytes.

Furthermore, every cell type has specific adhesion requirements. To provide a ready-to-use HIS contraction test, adhesion was optimized for card iomyocytes.

Furthermore, the periodic contraction/relaxation mode of cardiomyocytes is different from the isometric (long-lasting) contraction of fibroblasts, requiring a different detection and quantification approach with different time constraints.

### Protein Coating and Cell Concentration

In order for beating cardiomyocytes or colonies to wrinkle the biocompatible silicone elastomer film, force transmission must occur between the contracting cell and the film. Optimal force transmission is achieved by strongly attaching and spreading cardiomyocytes. If colonies grow in 3D cell clumps, forces developed during beating forces are insufficiently (if at all) transmitted and no wrinkles are formed in the film. To obtain monolayers or colonies of well-spread cardiomyocytes on the substrates with high wrinkling levels, the optimal surface treatment and protein coating was determined. A variety of cell adhesion compounds were tested, including but not restricted to fibronectin, gelatin, collagen type I/IV, vitronectin, pronectin, DOPA, and N-acetyl glucosamine (+combinations).

To meet standard requirements on product shelf life and ease-of-shipping, the surface treatment method was refined to provide the film surface with a dry layer of adhesive protein. The improved treatment process (“APTES/EDAC”) comprises sequential oxidation with plasma oxygen, 1% APTES for 90 min, 100 μg/ml 1-Ethyl-3-(3-dimethylaminopropyl) carbodiimide (EDAC) for 10 min (to replace the previous paraformaldehyde step), protein wet coating, and air drying. Fibronectin and gelatin were used as most widely applied proteins to promote cardiomyocyte adhesion.

Cardiomyocytes were seeded onto multi-well plates (as shown in FIG. 12) treated for 60 min with either fibronectin (2 μg/ml) or gelatin (2000, 200, 20, 2 μg/ml) with and without preceding APTES/EDAC functionalization. Spreading and adhesion of cells was assessed after 2 days by measuring the area of single cell spreading in each image and normalizing to the total area. Next, the average number of beating colonies per well was quantified for each coating condition and the percentage of beating colonies which were producing wrinkles was also quantified. Coating with fibronectin (2 μg/ml) and low concentrations of gelatin (2 and 20 μg/ml) achieved 80-90% well area coverage by spreading cardiomyocytes following APTES/EDAC treatment compared to lower cell spreading (75%) if the APTES/EDAC step was omitted.

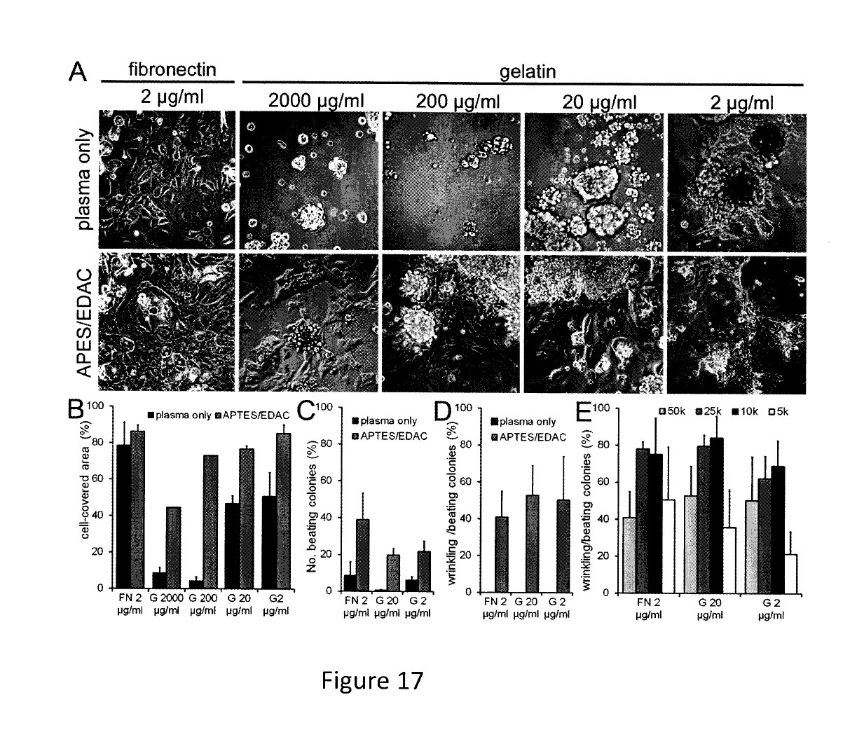

Referring to FIG. 17, hES2-derived cardiomyocytes were seeded in different concentrations on a biocompatible silicone elastomer film provided with and without APTES/EDAC treatment and matrix proteins in different concentrations. (A) Phase contrast images, (B) Quantification of cell covered area. (C) The average number of beating colonies per well and (D, E) the percentage of beating colonies creating wrinkles was quantified for fibronectin (FN 2 μg/ml) and gelatin (2 and 20 μg/ml)-coated wrinkling substrates. (E). To determine the optimal cell concentration for cardiomyocyte wrinkling, cells were seeded at 50,000, 25,000, 10,000 and 5,000 cells/cm² onto APTES/EDAC treated films and percentage of beating colonies creating wrinkles was quantified.

The number of beating colonies was at least 20-30/well across all protein coatings following APTES/EDAC treatment but <10 beating colonies/well without APTES/EDAC functionalization (FIG. 17C). APTES/EDAC functionalization obtained wrinkles which were visible in ˜50% of all beating colonies (FIG. 17D). APTES/EDAC treated surfaces with fibronectin (2 μg/ml) and gelatin (2 and 20 μg/ml) coating were selected to optimize cell densities for contraction analysis. Cardiomyocyte seeding densities were reduced from 50,000 to 25,000, 10,000 and 5,000 cells/cm² and wrinkling percentages were quantified. With cell density adaptation, the percentage of wrinkling among beating colonies reached 80-90% across all protein coatings with 10,000-25,000 cells/cm². Too confluent cardiomyocyte growth resulted in the formation of 3D aggregates with poor force transmission to the wrinkling substrates. Too low density reduced the number of beating colonies and percentage of wrinkling cells (FIG. 17E). For all further experiments, the plasma oxidized and APTES/EDAC-treated biocompatible silicone elastomers of 96-wells were coated with gelatin at a concentration of 2 μg/ml and provided with 10,000 cells/cm² before wrinkling was assessed after 2 days.

### Automated Cardiomyocyte Contraction Analysis

The periodic contraction/relaxation cycles of cardiomyocytes (tens of contractions per minute) differ from the isometric and hour-lasting contraction of fibroblasts and require higher frequency of image acquisition.

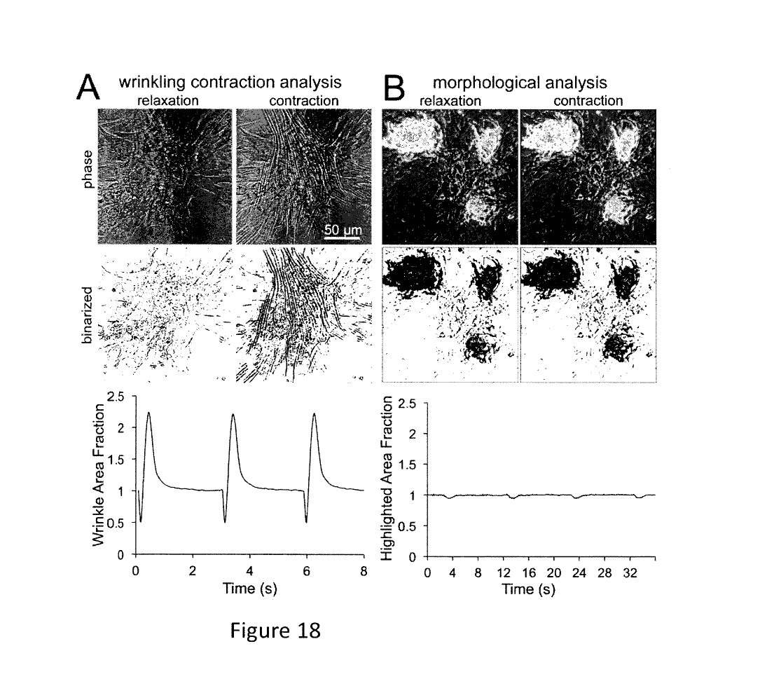

Cardiomyocyte contraction was assessed by recording image sequences over at least 15 s with an image acquisition rate of 10 frames/s. Acquisition was performed well-by-well on four image fields per well using the 10× objective of an inverted microscope with motorized stage and fully automated stage/acquisition control. The setup was chosen to provide conditions equivalent to commercially available HTS imaging stations. The periodic beating of cardiomyocytes over time allows direct comparison of the wrinkle signal in the contracted state with the resting state (FIG. 18) which reduces the impact of background signals (e.g., bright cell structures) that do not change over time. When comparing the signals generated by contracting cardiomyocytes on films with the competitive method of shape analysis on stiff culture substrates, wrinkling analysis delivered a 10-20-fold higher signal (FIG. 18).

FIG. 18 shows hES2-derived cardiomyocytes that were either cultured (A) in the wells of a device similar to that shown in FIG. 12, including a coating of a biocompatible silicone elastomer film having a surface that was oxidized with plasma oxidation and activated for cellular adhesion with APTES/EDAC and gelatin, and (B) gelatin coated culture plastic supports. Morphological analysis by thresholding, binarization, and area measurements of bright features (used in wrinkling analysis and commercial imaging systems to quantify cardiomyocyte beating) demonstrates dramatic contraction signal amplification on wrinkling substrates.

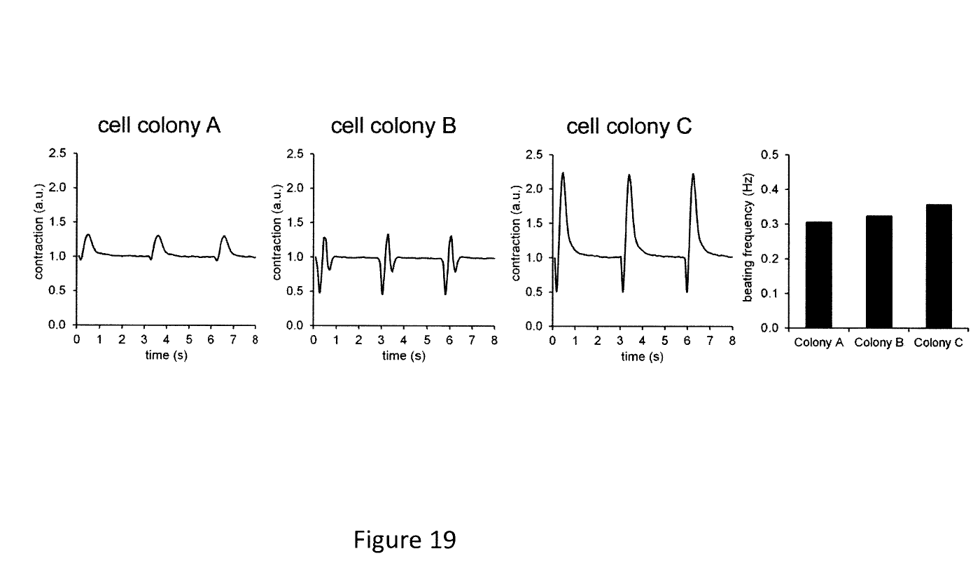

The devices and methods described herein may deliver clean frequency data of beating cardiomyocytes. In any given well, different numbers of cardiomyocytes will assemble in colonies to beat in synchronicity, resulting in different force amplitudes between different colonies (FIG. 19). However, the mechanical properties of the biocompatible silicone elastomer allow force transmission between adjacent colonies/cells over the elastomer, leading to an overall tendency of all cells/colonies within one well to beat synchronously (FIG. 19).

Referring to FIG. 19, cardiomyocyte colonies in close vicinity but physically separate were analyzed for wrinkle formation (contraction) using one well of a device similar to that shown in FIG. 12, including a biocompatible silicone elastomer film having a surface that was oxidized with plasma oxidation and activated for cellular adhesion with APTES/EDAC and gelatin. Beating frequency per colony was extracted using Fast Fourier analysis and compared.

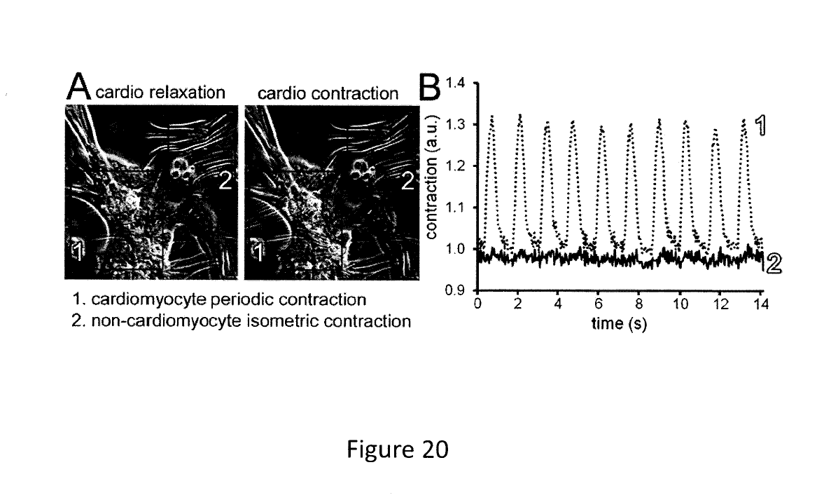

The fact that all colonies beat with similar frequency is advantageous for low magnification analysis, i.e. whole well imaging. Additionally, the contraction quantification allows to select different regions within one well to be analyzed separately. This feature may be used to investigate “arrhythmia” drug effects in vitro. To this end, proof-of-principle was provided by separately analyzing hES2s that differentiated into cardiomyocytes and hES2s that attained a fibroblastic phenotype (FIG. 20). Whereas cardiomyocytes exhibit the expected periodic contraction, the fibroblast-like cells exhibit isometric contraction that remains unchanged over the observation time of 15 s. Hence, in an overall analysis of the whole well, isometrically contracting cells (at least without added treatment) will not contribute to the contraction signal, further reducing background noise (FIG. 20).

The capability of the device shown in FIG. 12 will be further demonstrated to detect frequency changes, arrhythmia, and changes in contraction amplitudes in response to cardiomyocyte-affecting drugs. Benchmark drugs that are classically used to decrease frequency of beating dose-dependently include diltiazem, verapamil, procainamide, and felcainide (Yokoo et al., 2009). Conversely, adrenaline, isoproterenol, isobutyl methylxanthine, phenylephrine, and isoprenaline have been shown to increase beating frequency (Gai et al, 2009).

### Signal-to-Noise Ratio

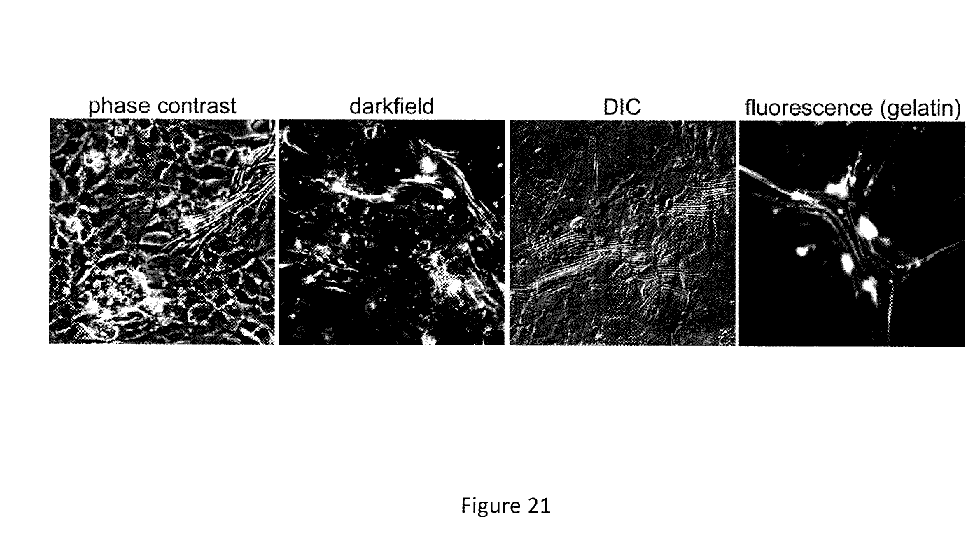

In sub-confluent fibroblast or cardiomyocyte cultures, transmission light (phase) images can provide an impeccable wrinkle/force signal. However, in confluent or over-confluent cardiomyocyte cultures, background may substantially increase. To specifically amplify wrinkle signals, a number of optical methods were tested that are available with HTS imaging stations. Referring to FIG. 21, hESC-derived cardiomyocytes were seeded onto a device similar to that shown in FIG. 12, having a surface that was oxidized via plasma oxidation and activated for adhesion with ATPES/EDAC and fluorescent gelatin. Cells and wrinkles were analyzed with different optical methods, including brightfield, dark field, and differential interference contrast (DIC) that are all compatible with wrinkle analysis.

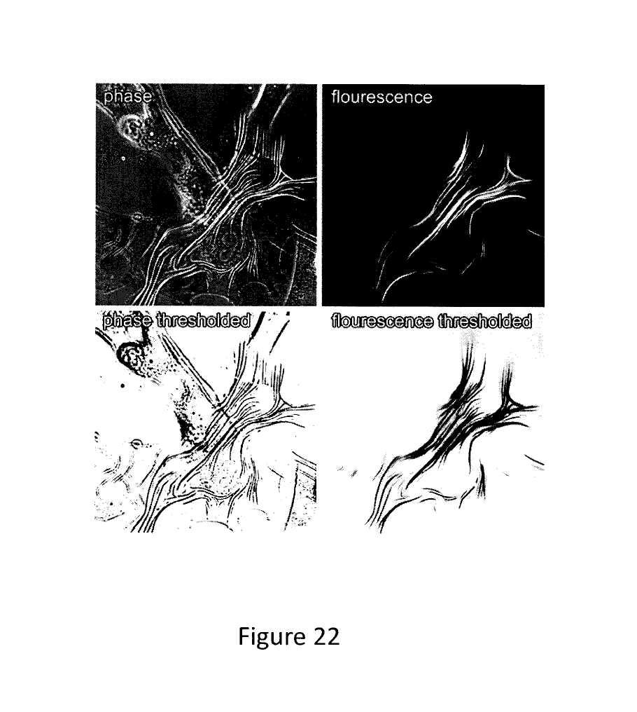

To further optimize the device of FIG. 12 for cardiomyocytes, new wrinkle detection approaches were developed. Particularly, wrinkles were assessed with epifluorescence. Results with surface-bound fluorescent dyes demonstrate feasibility of this approach. Referring to FIG. 22, fibroblasts were seeded onto a device similar to that shown in FIG. 12, having a surface that was oxidized via plasma oxidation and activated for adhesion with ATPES/EDAC and provided with a layer of fluorescently labelled fibronectin. The fluorescent signal was amplified by the formation of wrinkles and provides a cleaner signal after image binarization due to the fact that cell structures are not labeled.

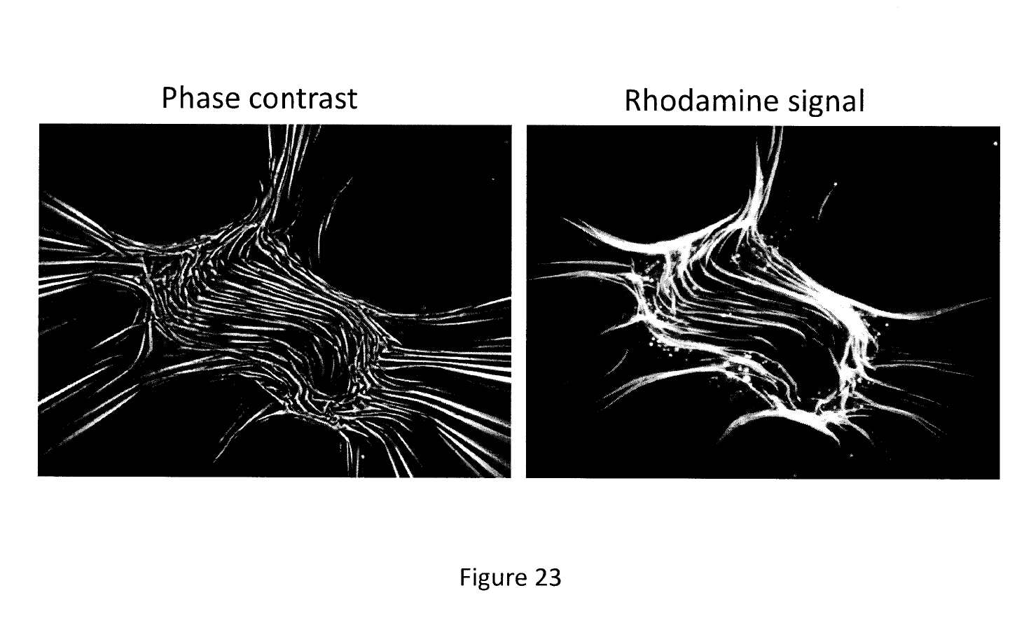

To further improve the surface labelling procedure to obtain signals that are sufficiently strong for detection in HTS epifluorescence imaging stations, fluorochromes were directly and covalently linked to the polymer surface. Referring to FIG. 23, after a sequence of silicone plasma oxygenation and treatment with APTES, amine groups may become available on the silicone surface to react with isothiocyanate (ITC)-functionalized Rhodamine (Rh-ITC). After the reaction, free carboxyl groups of the Rhodamine may react with amine groups of matrix proteins that are added to enhance cell adhesion. Cell attachment and viability were not affected by adding the Rh-ITC layer. FIG. 23 shows that the fluorescence intensity produced by Rh-ITC functionalized wrinkling substrates was sufficiently strong to detect fluorescent wrinkles with low resolution optics (20× air objective) and short camera exposure times (20 ms).

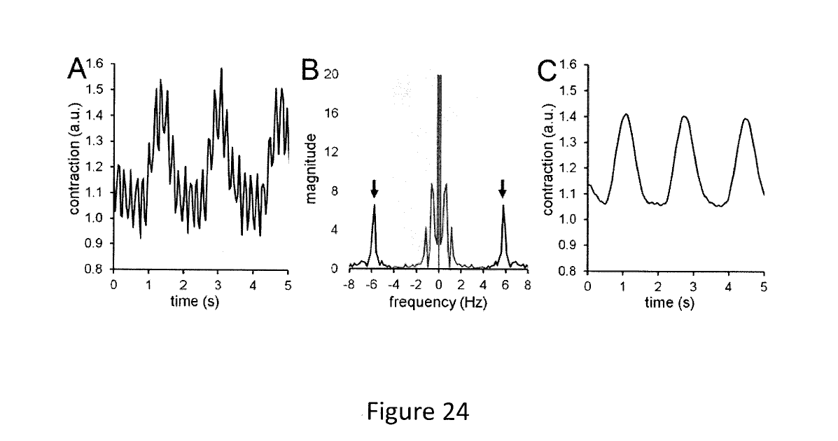

Different filtering procedures were also assessed. In one example high-frequency periodic noise was experimentally introduced to overlay the lower frequency of beating cardiomyocytes. Similar noises are frequently produced by imaging systems due to electrical noise and flickering lamps of the acquisition system. Using Fast Fourier analysis, the dominant frequencies can be automatically extracted and physiologically irrelevant signals can be eliminated by band-pass filtering. Referring to FIG. 24(A), the wrinkling-derived periodic signal of contracting cardiomyocytes was overlaid experimentally with periodic noise. Referring to FIG. 24(B), Fast Fourier filtering was used to determine the main frequencies (peaks) and band-pass filtering was applied to eliminate high frequency peaks (arrows). Referring to FIG. 24(C), the filtered signal does not contain the high frequency domain.

### Adapting Polymer Stiffness to the Mechanical Niche of Cardiomyocytes

The stiffness of the surface on which cells are grown is a powerful factor to determine cell behaviour and identity (Discher et al., 2009a, Discher et al., 2009b). Stiffness is measured as Young's elastic modulus E (in Pa), i.e., the force per area (stress) that is required to deform materials. Notably, cardiomyocytes spontaneously develop functional sarcomers and contract on heart-soft (10,000-20,000 Pa) substrates but not on stiff culture surfaces such as plastic (Engler et al., 2008, Chopra et al., 2011).

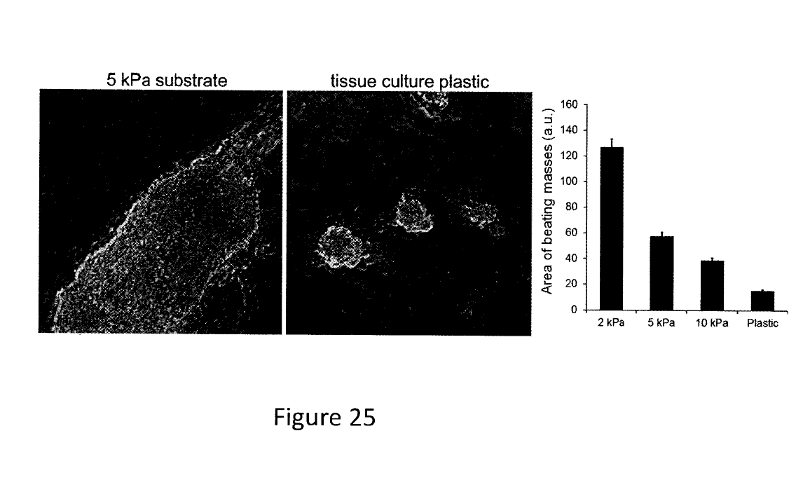

The optimal elastic modulus for the polymer to permit formation of visible surface wrinkles and reproduce a physiological mechanical environment for cardiomyocytes was determined and optimized. Referring to FIG. 25, hES2-derived cardiomyocytes were grown on biocompatible silicone elastomer films having a surface that was oxidized via plasma oxidation and activated for adhesion with ATPES/EDAC and gelatin, and on gelatin-coated tissue culture plastic. After 2 weeks, the surface area covered by periodically beating cell masses was quantified. After 2 weeks of growth of hES2-derived cardiomyocytes on gelatin-treated surfaces, the area covered by periodically beating cells masses was ˜10-times higher on the softest surface. For technical considerations and optimal wrinkle morphology, an elastic modulus of E=5,000-10,000 Pa was selected as optimal.

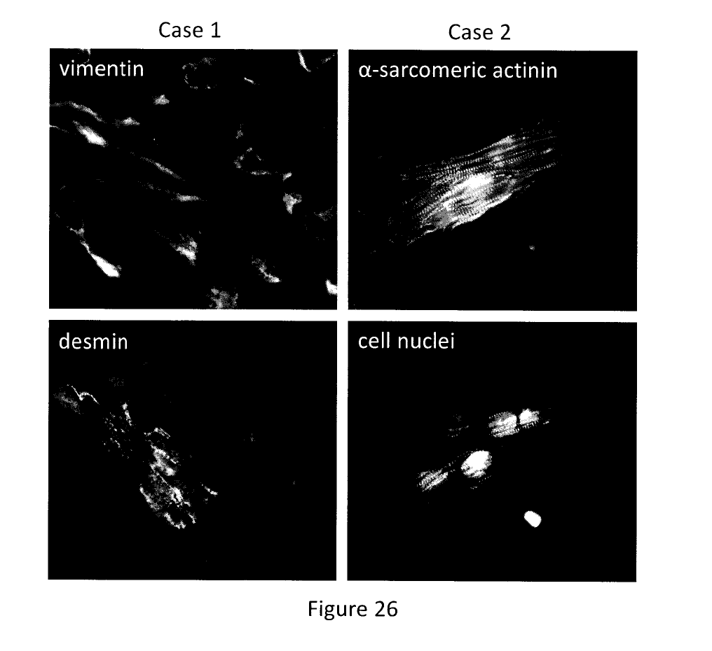

Referring to FIG. 26, hES2-derived cardiomyocytes were grown on biocompatible silicone elastomer films having a surface that was oxidized via plasma oxidation and activated for adhesion with ATPES/EDAC and gelatin. After 7 days of growth, 5,000 Pa soft wrinkling substrates stimulated formation of sarcomeric α-actinin- and desmin-positive cardiomyocyte colonies.

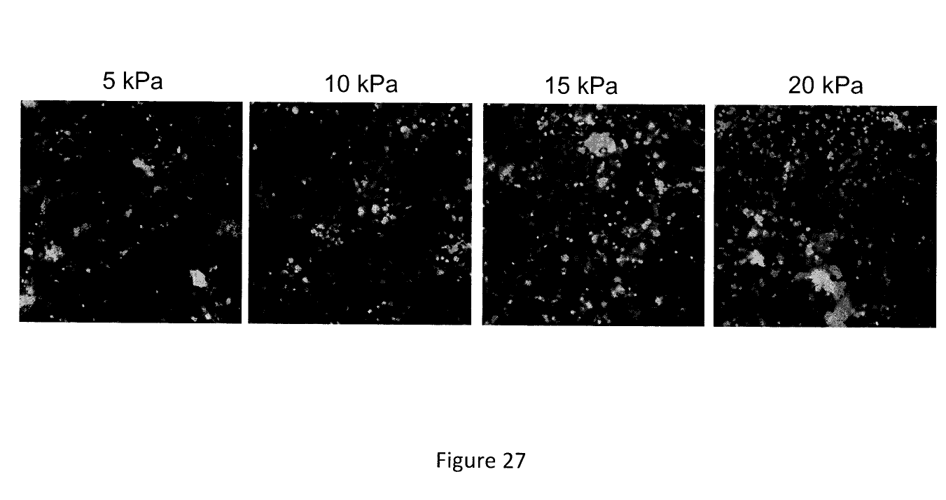

Referring to FIG. 27, hES2-derived cardiomyocytes were grown on biocompatible silicone elastomer films with modulus of 5,000 Pa, 10,000 Pa, 15,000 Pa, and 20,000 Pa, having a surface that was oxidized via plasma oxidation and activated for adhesion with ATPES/EDAC and gelatin. FIG. 27 shows that after 7 days of growth, 10,000 Pa soft wrinkling substrates favored formation of sarcomeric α-actinin-positive cardiomyocyte colonies whereas stiffer substrates selectively promoted the growth of α-actinin-negative fibroblastic cells.

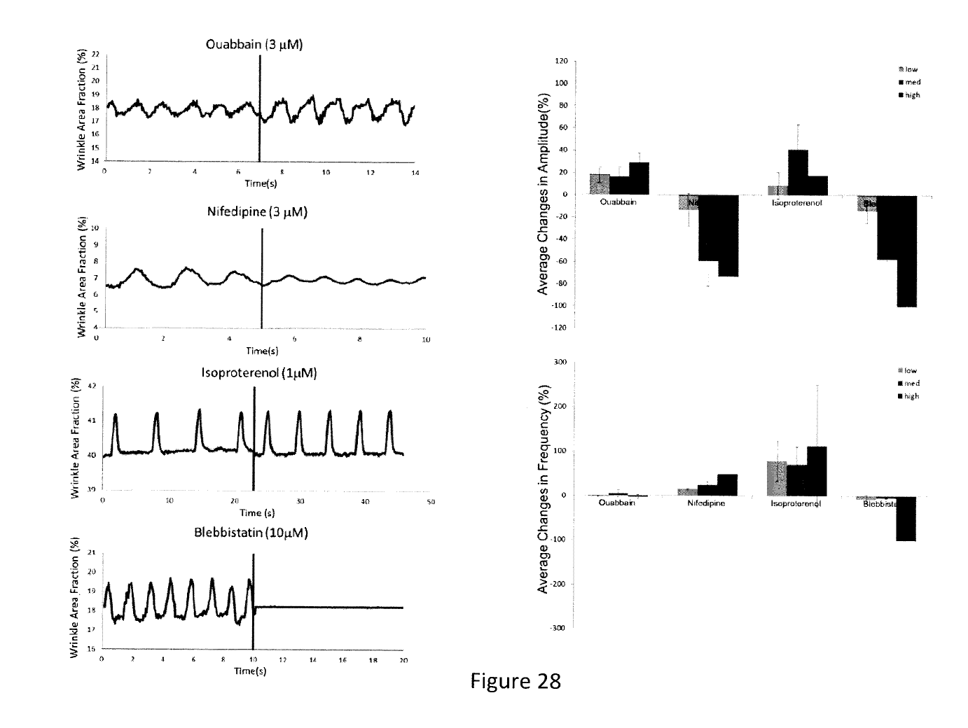

To demonstrate that the device of FIG. 12 has the capability to detect even small changes in cardiomyocyte contraction frequency, amplitude, and rhythmic contraction, hES2-derived cardiomyocytes were grown on biocompatible silicone elastomer films having a surface that was oxidized via plasma oxidation and activated for adhesion with ATPES/EDAC and gelatin. Referring to FIG. 28, periodically contracting cardiomyocyte colonies were recorded and live treated with cardiomyocyte affecting drugs in three concentrations (high, medium, low). Ouabain (0.3, 3, 30 μM) increased contraction amplitude at unchanged frequency, nifidepine (0.3, 3, 30 μM) decreased contraction amplitude and increased frequency, isoproterenol (0.1, 1, 10 μM) increased contraction frequency and amplitude, and blebbistatin (0.1, 1, 10 μM) decreased contraction amplitude and frequency.

### REFERENCES

1. Arora, P. D., N. Narani, and C. A. McCulloch. 1999. The compliance of collagen gels regulates transforming growth factor-beta induction of alpha-smooth muscle actin in fibroblasts. Am J Pathol. 154:871-882.
2. Balaban, N. Q., U. S. Schwarz, D. Riveline, P. Goichberg, G. Tzur, I. Sabanay, D. Mahalu, S. Safran, A. Bershadsky, L. Addadi, and B. Geiger. 2001. Force and focal adhesion assembly: a close relationship studied using elastic micropatterned substrates. Nat Cell Biol. 3:466-472.
3. Ballestrem, C., B. Hinz, B. A. Imhof, and B. Wehrle-Haller. 2001. Marching at the front and dragging behind: differential alphaVbeta3-integrin turnover regulates focal adhesion behavior. J Cell Biol. 155:1319-1332.
4. Beningo, K. A. & Wang, Y. L. Flexible substrata for the detection of cellular traction forces. Trends Cell Biol 12, 79-84 (2002).
5. Burton, K., J. H. Park, and D. L. Taylor. 1999. Keratocytes generate traction forces in two phases. Mol Biol Cell. 10:3745-3769.
6. Buxboim A, Rajagopal K, Brown A E, Discher D E. How deeply cells feel: Methods for thin gels. J Phys Condens Matter. 2010; 22:194116.
7. Chen, C. S., Tan, J. & Tien, J. Mechanotransduction at cell-matrix and cell-cell contacts. Annu Rev Biomed Eng 6, 275-302 (2004).
8. Chopra, A., Tabdanov, E., Patel, H., Janmey, P. A. & Kresh, J. Y. Cardiac myocyte remodeling mediated by N-cadherin-dependent mechanosensing. Am J Physiol Heart Circ Physiol 300, H1252-1266 (2011).
9. Clement, S., B. Hinz, V. Dugina, G. Gabbiani, and C. Chaponnier. 2005. The N-terminal Ac-EEED sequence plays a role in {alpha}-smooth-muscle actin incorporation into stress fibers. J Cell Sci. 118:1395-1404.
10. Cukierman, E., R. Pankov, and K. M. Yamada. 2002. Cell interactions with three-dimensional matrices. Curr Opin Cell Biol. 14:633-639.
11. Dimitriadis, E. K., F. Horkay, J. Maresca, B. Kachar, and R. S. Chadwick. 2002. Determination of elastic moduli of thin layers of soft material using the atomic force microscope. Biophys J. 82:2798-2810.
12. Discher, D. E., Mooney, D. J. & Zandstra, P. W. Growth factors, matrices, and forces combine and control stem cells. Science 324, 1673-1677 (2009a).
13. Discher, D. et al. Biomechanics: cell research and applications for the next decade. Ann Biomed Eng 37, 847-859 (2009b).
14. Engler, A. J. et al. Embryonic cardiomyocytes beat best on a matrix with heart-like elasticity: scar-like rigidity inhibits beating. J Cell Sci 121, 3794-3802 (2008).
15. Engler, A. J., L. Richert, J. Y. Wong, C. Picart, and D. E. Discher. 2004b. Surface probe measurements of the elasticity of sectioned tissue, thin gels and polyelectrolyte multilayer films: Correlations between substrate stiffness and cell adhesion. Surface Science. 570:142-154.
16. Ernsting, M. J., Bonin C. G., Yang, M., Labow, R. S., Santerre, J. P. 2005. Generation of Cell Adhesive Substrates Using Peptide Fluoroalkyl Surface Modifiers. Biomaterials. 26(33):6536-46.
17. Gai, H. et al. Generation and characterization of functional cardiomyocytes using induced pluripotent stem cells derived from human fibroblasts. Cell Biol Int 33, 1184-1193 (2009).
18. Geiger, B., A. Bershadsky, R. Pankov, and K. M. Yamada. 2001. Transmembrane crosstalk between the extracellular matrix—cytoskeleton crosstalk. Nat Rev Mol Cell Biol. 2:793-805.
19. Goffin, J. M., P. Pittet, G. Csucs, J. W. Lussi, J. J. Meister, and B. Hinz. 2006. Focal adhesion size controls tension-dependent recruitment of alpha-smooth muscle actin to stress fibers. J Cell Biol. 172:259-268.
20. Harris, A. K., Wild, P. & Stopak, D. Silicone rubber substrata: a new wrinkle in the study of cell locomotion. Science 208, 177-179 (1980).
21. Hinz B. The myofibroblast: Paradigm for a mechanically active cell. J Biomech. 2010; 43:146-155
22. Hinz, B., and G. Gabbiani. 2003. Mechanisms of force generation and transmission by myofibroblasts. Curr Opin Biotechnol. 14:538-546.
23. Hinz, B., G. Gabbiani, and C. Chaponnier. 2002. The NH2-terminal peptide of alpha-smooth muscle actin inhibits force generation by the myofibroblast in vitro and in vivo. J Cell Biol. 157:657-663.
24. Hinz, B., G. Celetta, J. J. Tomasek, G. Gabbiani, and C. Chaponnier. 2001a. Alpha-smooth muscle actin expression upregulates fibroblast contractile activity. Mol Biol Cell. 12:2730-2741.
25. Hinz, B., W. Alt, C. Johnen, V. Herzog, and H. W. Kaiser. 1999. Quantifying lamella dynamics of cultured cells by SACED, a new computer-assisted motion analysis. Exp Cell Res. 251:234-243.
26. Klingberg F, Hinz B, White E S. The myofibroblast matrix: Implications for tissue repair and fibrosis. J Pathol. 2013; 229:298-309
27. Lee, J. N., X. Jiang, D. Ryan, and G. M. Whitesides. 2004. Compatibility of mammalian cells on surfaces of poly(dimethylsiloxane). Langmuir. 20:11684-11691.
28. Lee, J., M. Leonard, T. Oliver, A. Ishihara, and K. Jacobson. 1994. Traction forces generated by locomoting keratocytes. J Cell Biol. 127:1957-1964.
29. Pelham, R. J., Jr. & Wang, Y. Cell locomotion and focal adhesions are regulated by substrate flexibility. Proc Natl Acad Sci USA 94, 13661-13665 (1997).
30. Skalli, O., P. Ropraz, A. Trzeciak, G. Benzonana, D. Gillessen, and G. Gabbiani. 1986. A monoclonal antibody against alpha-smooth muscle actin: a new probe for smooth muscle differentiation. J Cell Biol. 103:2787-2796.
31. Wynn T A. Cellular and molecular mechanisms of fibrosis. J Pathol. 2008; 214:199-210
32. Yeung, T., P. C. Georges, L. A. Flanagan, B. Marg, M. Ortiz, M. Funaki, N. Zahir, W. Ming, V. Weaver, and P. A. Janmey. 2005. Effects of substrate stiffness on cell morphology, cytoskeletal structure, and adhesion. Cell Motil Cytoskeleton. 60:24-34.
33. Yokoo, N. et al. The effects of cardioactive drugs on cardiomyocytes derived from human induced pluripotent stem cells. Biochem Biophys Res Commun 387, 482-488 (2009).
34. Zamir, E., M. Katz, Y. Posen, N. Erez, K. M. Yamada, B. Z. Katz, S. Lin, D. C. Lin, A. Bershadsky, Z. Kam, and B. Geiger. 2000. Dynamics and segregation of cell-matrix adhesions in cultured fibroblasts. Nat Cell Biol. 2:191-196.

1. A method for assessing cell contraction, the method comprising:
a) adhering contractile cells to an oxidized and cellular adhesion activated surface of a biocompatible silicone elastomer film, wherein the biocompatible silicone elastomer film allows the cells to contract and wrinkles when the cells contract; and
b) analyzing wrinkles in the biocompatible silicone elastomer film formed by contraction of the contractile cells.

2. The method of claim 1, wherein the contractile cells are at least one of fibroblasts, myofibroblasts, epithelial cells, endothelial cells, cardiomyocytes, skeletal muscle cells, smooth muscle cells, mesenchymal stem cells, induced pluripotent stem cells, embryonic stem cells, inflammatory cells, cancer cells, immortalized lineage cells, hepatic stellate cells, pericytes, chondrocytes, chondroblasts, osteoblasts, osteoclasts, astrocytes, myoepithelial cells, glial cells, and neuronal cells.

3. The method of claim 1, wherein the contractile cells are fibroblasts.

4. The method of claim 1, wherein the contractile cells are cardiomyocytes.

5. The method of claim 1, wherein the cells are in the form of a tissue, and the tissue is at least one of fibrotic tissue, scar tissue, heart muscle tissue, skeletal muscle tissue, smooth muscle tissue, arterial tissue, venous tissue, connective tissue, nervous tissue, liver tissue, kidney tissue, lung tissue, gastrointestinal tissue, cancer tissue, bone marrow tissue, blood tissue, cartilage tissue, bone tissue, gingiva tissue, skin tissue, tendon tissue, fascia tissue, glandular tissue, embryonic tissue, and reproductive tissue.

6. The method of any one of claims 1 to 5, wherein the biocompatible silicone elastomer film is fully polymerized.

7. The method of any one of claims 1 to 6, wherein the biocompatible silicone elastomer is a polydimethylsiloxane.

8. The method of any one of claims 1 to 7, wherein step b) comprises:
i) obtaining an image of the biocompatible silicone elastomer film;
ii) determining a proportion of the image that contains the wrinkles; and
iii) comparing the proportion to a control.

9. The method of any one of claims 1 to 7, wherein step b) is performed via live imaging.

10. The method of any one of claims 1 to 7, wherein step b) is performed in real time.

11. The method of any one of claims 1 to 10, wherein prior to step a), the method further comprises oxidizing the surface of a raw biocompatible silicone elastomer film, and activating the oxidized surface for cellular adhesion, to yield the oxidized and cellular adhesion activated surface.

12. The method of claim 11, wherein oxidizing the surface comprises plasma oxidation of the surface.

13. The method of claim 11, wherein oxidizing the surface comprises treating the surface with hydrogen peroxide and sulfuric acid.

14. The method of any one of claims 11 to 13, wherein activating the oxidized surface for cellular adhesion comprises:
i) silanizing the oxidized surface; and
ii) treating the oxidized surface with an extracellular matrix (ECM) protein.

15. The method of claim 14, further comprising fluorescently labeling the ECM protein.

16. The method of claim 14, wherein silanizing the oxidized surface comprises treating the oxidized surface with 3-aminopropyltriethoxysilane (APTES).

17. The method of claim 16, wherein silanizing the oxidized surface further comprises treating the oxidized surface with at least one of paraformaldehyde and 1-ethyl-3-(3-dimethylaminopropyl) carbodiimide (EDAC) subsequent to treatment with APTES.

18. The method of claim 16, wherein silanizing the oxidized surface further comprises treating the oxidized surface with reactive fluorochromes subsequent to treatment with APTES.

19. The method of claim 18, wherein a reactive moiety of the fluorochromes comprises isotyocyanate (ITCO) and the fluorochrome comprises rhodamine.

20. The method of any one of claims 14 to 19, wherein the ECM protein includes at least one of gelatin, collagen, fibronectin, vitronectin, pronectin, DOPA, N-acetyl glucosamine, BSA, laminin, RGD peptides and derivatives, and combinations thereof.

21. The method of any one of claims 1 to 19, wherein the ECM protein is collagen.

22. The method of any one of claims 1 to 19, wherein the ECM protein is gelatin.

23. The method of any one of claims 1 to 22 wherein prior to step b), the method further comprises treating the cells to one of:
i) induce contraction; and
ii) inhibit contraction.

24. The method of any one of claims 1 to 23, wherein prior to step b), the method comprises treating the cells with a test compound, and step b) comprises assessing the effect of the test compound on cell contraction.

25. The method of claim 24, further comprising:
i) identifying the test compound as one of an inducer of contraction and an inhibitor of contraction; and
ii) if the test compound is identified as an inducer of contraction, selecting the test compound as a candidate treatment for at least one of chronic wound healing, low vascular tone, and arrhythmia.

26. The method of claim 24, further comprising:
i) identifying the test compound as one of an inducer of contraction and an inhibitor of contraction; and
ii) if the test compound is identified as an inhibitor of contraction, selecting the test compound as a candidate treatment for at least one of fibrocontractive disease, and cancer.

27. The method of any one of claims 1 to 26, further comprising selecting highly contractile cells of the contractile cells based on the analysis of step b) for purposes of autologous cell selection for cell therapies.

28. The method of claim 27, further comprising transplanting the highly contractile cells into a patient for cell therapy.

29. The method of any one of claims 1 to 28, wherein the film comprises a fluorescent dye, and step b) comprises imaging the wrinkles with fluorescence microscopy.

30. The method of any one of claims 1 to 29, wherein the contractile cells are cardiomyocytes, and step b) comprises quantifying a percentage of the contractile cells that are beating.

31. The method of any one of claims 1 to 30, wherein step b) comprises determining a contractile force of the cells.

32. The method of any one of claims 1 to 31, wherein the cells are cardiomyocytes, and step b) comprises determining a beating rate of the cells.

33. The method of any one of claims 1 to 32, further comprising blending a cell adhesive peptide coupled to a bioactive fluorinated surface modifier (BFSM) into the biocompatible silicone elastomer film.

34. The method of any one of claims 1 to 33, further comprising embedding a position marker in the biocompatible silicone elastomer film.

35. The method of any one of claims 1 to 34, wherein the biocompatible silicone elastomer film has a modulus of elasticity of between 0.5 kPa and 25 kPa.

36. The method of any one of claims 1 to 35, wherein the biocompatible silicone elastomer film has a modulus of elasticity of between 1.5 kPa and 3 kPa.

37. The method of any one of claims 1 to 36, wherein the biocompatible silicone elastomer film has a modulus of elasticity of about 5 kPa.

38. The method of any one of claims 1 to 37, wherein the film has a film thickness of less than 200 microns.

39. The method of any one of claims 1 to 38, wherein the film has a film thickness of between 20 microns and 40 microns.

40. The method of any one of claims 1 to 39, wherein the film has a film thickness of approximately 30 microns.

41. A device for assessing cell contraction comprising:
a) a plate comprising at least one well, each well having a well sidewall and a planar well bottom;
b) each well bottom comprising a coating of a biocompatible silicone elastomer film having an oxidized and cellular adhesion activated surface.

42. The device of claim 41, wherein the film has a film thickness of less than 200 microns.

43. The device of 41, wherein the film has a film thickness of between 20 microns and 40 microns.

44. The device of claim 41, wherein the film has a film thickness of approximately 30 microns.

45. The device of any one of claims 41 to 44, wherein the plate comprises:
a) an upper plate comprising at least one bottomless well, each bottomless well defining one of the well sidewalls;
b) a base plate formed separately from the upper plate and secured to the upper plate, the base plate comprising a planar face coated with the biocompatible silicone elastomer film to form the well bottoms.

46. The device of claim 45, wherein the base plate has a thickness of between 100 microns and 200 microns.

47. The device of any one of claims 45 and 46, wherein the base plate has a thickness of about 150 microns.

48. The device of any one of claims 45 to 47, wherein the base plate is transparent.

49. The device of any one of claims 45 to 47, wherein the base plate is fabricated from glass or tissue culture plastic.

50. The device of any one of claims 45 to 49, wherein the upper plate is fabricated from polystyrene.

51. The device of any one of claims 41 to 50, wherein the plate comprises a plurality of wells.

52. The device of any one of claims 41 to 51, wherein the plate comprises 96 wells.

53. The device of any one of claims 41 to 51, wherein the plate comprises 384 wells.

54. The device of any one of claims 41 to 53, wherein the biocompatible silicone elastomer film comprises a polydimethylsiloxane.

55. The device of any one of claims 41 to 54, wherein the oxidized and cellular adhesion activated surface comprises an extracellular matrix (ECM) protein, and the ECM protein is at least one of gelatin, collagen, fibronectin, vitronectin, pronectin, DOPA, N-acetyl glucosamine, BSA, laminin, RGD peptides and derivatives, and combinations thereof.

56. The device of claim 55, wherein the ECM protein is collagen.

57. The device of claim 55, wherein the ECM protein is gelatin.

58. The device of claim 55, wherein the ECM protein is fluorescently labeled.

59. The device of any one of claims 41 to 58, wherein the biocompatible silicone elastomer film comprises a fluorescent dye.

60. The device of any one of claims 41 to 59, wherein the device further comprises at least one position marker in the biocompatible silicone elastomer film.

61. The device of any one of claims 41 to 60, wherein the biocompatible silicone elastomer film has a modulus of elasticity of between 0.5 kPa and 25 kPa.

62. The device of any one of claims 41 to 61, wherein the biocompatible silicone elastomer film has a modulus of elasticity of between 1.5 kPa and 3.0 kPa.

63. The device of claim 62, wherein the biocompatible silicone elastomer film has a modulus of elasticity of about 5 kPa.

64. The device of any one of claims 41 to 63, wherein the biocompatible silicone elastomer film is transparent.

65. A method for fabricating a cell contraction assessment device, comprising:
a) coating a planar face of a base plate with a raw biocompatible silicone elastomer film;
b) oxidizing a surface of the a raw biocompatible silicone elastomer film, and activating a surface of the oxidized biocompatible silicone elastomer film for cellular adhesion, to yield a biocompatible silicone elastomer film having an oxidized and cellular adhesion activated surface;
c) securing the base plate to an upper plate comprising at least one bottomless well, whereby the bottomless well and base plate together form at least one well, the at least one well having a sidewall formed by the at least one bottomless well of the upper plate, and a well bottom formed by the base plate and the biocompatible silicone elastomer film.

66. The method of claim 65, wherein step a) comprises coating the planar face of the base plate with the raw biocompatible silicone elastomer film to yield a film thickness of less than 200 microns.

67. The method of claim 66, wherein step a) comprises coating the planar face of the base plate with the raw biocompatible silicone elastomer film to yield a film thickness of between 20 microns and 40 microns.

68. The method of claim 67, wherein step a) comprises coating the planar face of the base plate with the raw biocompatible silicone elastomer film to yield a film thickness of approximately 30 microns.

69. The method of any one of claims 65 to 68, wherein step b) comprises plasma oxidation of the surface of the raw biocompatible silicone elastomer film.

70. The method of any one of claims 65 to 68, wherein step b) comprises treating the surface of the raw biocompatible silicone elastomer film with hydrogen peroxide and sulfuric acid.

71. The method of any one of claims 65 to 70, wherein step a) comprises:
i) silanizing the surface of the oxidized biocompatible silicone elastomer film; and
ii) treating the surface of the oxidized biocompatible silicone elastomer film with an extracellular matrix (ECM) protein.

72. The method of claim 71, wherein the ECM protein is at least one of gelatin, collagen, fibronectin, vitronectin, pronectin, DOPA, N-acetyl glucosamine, BSA, laminin, RGD peptides and derivatives, and combinations thereof.

73. The method of claim 71, wherein the ECM protein is collagen.

74. The method of claim 71, wherein the ECM protein is gelatin.

75. The method of any one of claims 71 to 74, wherein the ECM protein is fluorescently labeled.

76. The method of any one of claims 71 to 75, wherein silanizing the surface of the oxidized biocompatible silicone elastomer film comprises treating the surface of the oxidized biocompatible silicone elastomer film with 3-aminopropyltriethoxysilane (APTES).

77. The method of claim 76, wherein silanizing the surface of the oxidized biocompatible silicone elastomer film further comprises treating the surface of the oxidized biocompatible silicone elastomer film with at least one of paraformaldehyde and 1-ethyl-3-(3-dimethylaminopropyl) carbodiimide (EDAC) subsequent to treatment with APTES.

78. The method of claim 76, wherein silanizing the oxidized surface further comprises treating the oxidized surface with reactive fluorochromes subsequent to treatment with APTES.

79. The method of claim 78, wherein a reactive moiety of the fluorochromes comprises isotyocyanate (ITCO) and the fluorochrome comprises rhodamine.

80. The method of any one of claims 65 to 79, wherein step b) comprises blending a cell adhesive peptide coupled to a bioactive fluorinated surface modifier (BFSM) into the raw biocompatible silicone elastomer film.

81. The method of any one of claims 65 to 80, wherein the raw biocompatible silicone elastomer film comprises a polydimethylsiloxane.

82. The method of any one of claims 65 to 81, wherein the base plate has a thickness of between 100 microns and 200 microns.

83. The method of claim 82, wherein the base plate has a thickness of about 150 microns.

84. The method of any one of claims 65 to 83, wherein the base plate is transparent.

85. The method of any one of claims 65 to 84 wherein the base plate is fabricated from glass or tissue culture plastic.

86. The method of any one of claims 65 to 85, wherein the upper plate is fabricated from polystyrene.

87. The method of any one of claims 65 to 86, wherein the upper plate comprises a plurality of wells.

88. The method of any one of claims 65 to 87, wherein the upper plate comprises 96 bottomless wells.

89. The method of any one of claims 65 to 87, wherein the upper plate comprises 384 bottomless wells.

90. The method of any one of claims 65 to 89, wherein step c) comprises clamping the upper plate to the base plate.

91. The method of any one of claims 65 to 90, wherein step a) comprises spin casting the raw biocompatible silicone elastomer film onto the planar face.

92. The method of any one of claims 65 to 91, further comprising embedding a position marker in the raw biocompatible silicone elastomer film.

93. The method of any one of claims 65 to 92, further comprising incorporating a fluorescent dye into the raw biocompatible silicone elastomer film.

94. The method of any one of claims 65 to 93, wherein the biocompatible silicone elastomer film has a modulus of elasticity of between 0.5 kPa and 25 kPa.

95. The method of any one of claims 65 to 94, wherein the biocompatible silicone elastomer film has a modulus of elasticity of between 1.5 kPa and 3.0 kPa.

96. The method of claim 95, wherein the biocompatible silicone elastomer film has a modulus of elasticity of about 5 kPa.

97. The method of any one of claims 65 to 96, wherein the biocompatible silicone elastomer film is transparent.

98. A method for assessing cell contraction using the device of any one of claims 41 to 64, the method comprising:
a) adhering contractile cells to the oxidized and cellular adhesion activated surface, wherein the films allow the cells to contract and wrinkle when the cells contract; and
b) analyzing wrinkles in the biocompatible silicone elastomer film formed by contraction of the contractile cells.
